(19) United States
(12) Patent Application Publication
(10) Pub. No.: US 2005/0170435 A1
(43) Pub. Date: Aug. 4, 2005

(54) Biosensor and use thereof to identify therapeutic drug molecules and molecules binding orphan receptors

(76) Inventors: Narasimhan Gautam, Saint Louis, MO (US); Muslum Akgoz, Corum (TR); Inaki Azpiazu, Saint Louis, MO (US)

Correspondence Address: Patrick W. Rasche;Armstrong Teasdale LLP, One Metropolitan Square, Suite 2600, St. Louis, MO, 63102, US

(21) Appl. No.: 10/914,049

(22) Filed: Aug. 7, 2004

Related U.S. Application Data
(63) Continuation-in-part of application No. 10/771,897, filed on Feb. 4, 2004.
(60) Provisional application No. 60/592,796, filed on Jul. 30, 2004.
(60) Provisional application No. 60/577,448, filed on Jun. 4, 2004.

(51) Int. Cl. G01N 33/53; G01N 33/567; C12N 5/06

(52) U.S. Cl. 435/7.2; 435/325

(57) ABSTRACT

A G protein biosensor cell comprises G protein beta, gamma or both beta and gamma subunits tagged with a fluorescent protein(s) expressed in living intact functional cells. The subcellular location of the fluorescent protein tagged beta, gamma or both beta and gamma subunits is strongly responsive to the activation state of specific G protein coupled receptors in the biosensor cell. The biosensor cell responds reproducibly to agonist and antagonist drug molecules specific for G protein coupled receptors by demonstrating translocation of the fluorescent protein tagged beta, gamma or both beta and gamma subunits from one part of the cell to another. The biosensor cells have utility in identifying and classifying candidate therapeutic drugs as to their therapeutic value.

This application is a CIP of pending U.S. application Ser. No. 10/771, 897 filed Feb. 4, 2004 titled “BIOSENSOR AND USE THEREOF TO IDENTIFY THERAPEUTIC DRUG MOLECULES AND MOLECULES BINDING ORPHAN RECEPTORS”. This application claims the benefit of U.S. provisional application Ser. No. 60/577,448 filed Jun. 4, 2004 titled “BIOSENSOR AND USE THEREOF TO IDENTIFY THERAPEUTIC DRUG MOLECULES AND MOLECULES BINDING ORPHAN RECEPTORS” which is incorporated herein in its entirety by reference. This application claims the benefit of U.S. provisional application Attorney Docket No.15060-82 filed Jul. 30, 2004 titled “BIOSENSOR AND USE THEREOF TO IDENTIFY THERAPEUTIC DRUG MOLECULES AND MOLECULES BINDING ORPHAN RECEPTORS” which is incorporated herein in its entirety by reference.

### STATEMENT REGARDING FEDERALLY SPONOSRED RESEARCH AND DEVELOPMENT

This invention was made with government support under Grant Number GM46963 and GM069027 awarded by the National Institute of Health and a post doctoral fellowship from American Heart Association 225378Z. The government has certain rights in the invention.

### FIELD OF THE INVENTION

This invention relates to recombinant DNA technology and the preparation and operation of a functional biosensor capable of and capably operating in a living intact functional cell. More particularly, this invention relates to G protein coupled receptors and to a method of screening for candidate molecules specifically binding to these receptors by non-invasively using a functional biosensor cell comprising G protein subunits in live intact cells to identify and classify candidate therapeutic drug molecules and to identify potential therapeutic efficacy.

### BACKGROUND

G proteins and their receptors play a key role in regulating cellular physiology. Some of the regulatory signaling pathways mediated by receptors and G proteins are implicated in the onset and progression of serious and fatal human diseases. G proteins comprise an alpha subunit and a betagamma subunit complex. G proteins are signal transducers—that is they mediate the conversion of an extracellular signal into an intracellular physiological response. On sensing a hormone, neurotransmitter, a natural or chemically synthesized agonist, an excited receptor activates a G protein resulting in the activation of the alpha subunit and betagamma subunit complex which subsequently regulate the function of effectors inside the cell. (See also Molecular Biology of the Cell, 4th Edition, Alberts and others, Garland Science, N.Y., in particular Chapter 15 thereof, including pages 852-856).

In live mammalian systems such as human, rat and mice, G protein signaling pathways are extraordinarily complex compared to G protein signaling pathways in single cell organisms such as yeast (Saccharomyces cerevisiae) and soil amoeba (Dictyostelium discoideum). Yeast and soil amoeba cells contain a few G protein coupled receptor types and G protein types while in contrast mammalian cells contain hundreds of G protein coupled receptor types and a large variety of G protein subunit types.

Many of the molecular mechanisms underlying G protein signaling pathways have so far been elucidated in in vitro systems using purified proteins and broken cells. However, G protein signaling functions occur in intact living cells subject to constraints of dynamic equilibrium, which are disrupted when cells are broken.

Additionally, as mentioned before, mammalian cells contain large families of G protein subunits, receptors and effector molecules leading to the generation of vast networks of membrane transduction signaling pathways which are functional only when the cell is intact and living. Unfortunately, relatively little information is at present available about the behavior of these signaling pathways in an intact living mammalian cell because methods have not been available for their observation.

Several mechanisms at the basis of G protein signaling have been identified so far. Results have shown that receptor stimulated dissociation of the G protein subunits leads to the activation of effectors downstream and thus signaling pathways. Both activated subunits, the GTP bound alpha subunit and the betagamma complex, act on effector molecules. Subsequent formation of the G protein heterotrimer as a result of receptor inactivation, switches off effector signaling activity of the G protein subunits. In order to elucidate more information, soil amoeba (D. discoideum) G protein subunits have been labeled with fluorescent proteins and expressed in soil amoeba (D. discoideum) cells providing the capability of detecting a fluorescence signal emanating from a heterotrimer and detecting the loss of fluorescence signal upon activation of the heterotrimer.

G protein coupled receptors form the single largest target for commercially available pharmaceutical drugs today. It is estimated that fifty percent of recently launched drugs were targeted at these receptors with annual worldwide sales exceeding about $30 billion in year 2001. Among the one hundred highest selling drugs, about 25% were directed at G protein coupled receptors.

However, today's available commercial drugs are targeted at a relatively small proportion of known G protein coupled receptors.

While the three dimensional structure of the G protein coupled receptor and newer methods of rational drug design increase the range and depth of candidate molecules that are available, there is at present an undesired serious limitation in methods available to screen drug candidates non-invasively using mammalian G protein coupled receptors and G proteins.

There is also a lack of information about the temporal changes and spatial localization of the effects of candidate therapeutic molecules in an intact living cell.

### BRIEF DESCRIPTION OF THE INVENTION

In a first aspect, a functional biosensor comprises a G protein signaling subunit(s) fused to a fluorescent protein or a luminescent protein.

In an aspect, a live functional G protein biosensor cell comprises a G protein beta or gamma subunit or both subunits tagged with a fluorescent protein or a luminescent protein.

In an aspect, a live functional G protein biosensor cell comprises an endogenous or introduced G protein alpha subunit and introduced beta and gamma subunits one of which or both of which are tagged with a fluorescent or luminescent protein.

In an aspect, a live functional G protein biosensor cell comprises an endogenous or introduced G protein alpha subunit and an introduced gamma subunit tagged with a fluorescent or luminescent protein with an endogenous beta subunit.

In an aspect, a live functional G protein biosensor cell comprises an endogenous or introduced G protein alpha subunit and an introduced beta subunit tagged with a fluorescent or luminescent protein with an endogenous gamma subunit.

In an aspect, a screening method for screening natural or chemically synthesized candidate agonists and antagonists that bind to previously characterized, uncharacterized or “orphan” mammalian receptors comprising the operation pf an intact living cell containing said receptors and fluorescent protein or luminescent protein tagged G protein beta subunit, gamma subunit or beta and gamma subunits which when exposed to said candidate agonists elicits the translocation of the tagged beta subunit, gamma subunit or beta and gamma subunits from the plasma membrane to the cell interior and which when exposed subsequently to an antagonist results in the translocation of the tagged beta subunit, gamma subunit or beta and gamma subunits from the cell interior to the plasma membrane of the cell thereby identifying respective agonist(s) and antagonist(s) for characterized, uncharacterized or orphan receptors.

In an aspect, exposure to an antagonist follows exposure to an agonist and in another aspect exposure to the agonist in the presence of the antagonist prevents translocation of the beta, gamma or beta and gamma subunits.

In an aspect, a non-invasive method for identifying a candidate therapeutic drug molecule by obtaining images of the cell over a time period from a live functional biosensor cell comprising a G protein beta subunit, gamma subunit or beta and gamma subunits tagged with a fluorescent or luminescent protein and a known receptor or an orphan receptor (a) in the absence of an added candidate molecule, (b) in the presence of an added molecule and then comparing the images of (b) with the images of (a) visually or by using appropriate image analysis computing software to determine whether images from (b) demonstrate translocation of the beta subunit, gamma subunit or beta and gamma subunits from the plasma membrane to cell interior or translocation from the cell interior to the plasma membrane of the cell.

In an aspect, a non-invasive method for identifying a candidate therapeutic drug molecule by obtaining images of the cell over a time period from a live functional biosensor cell comprising a G protein alpha subunit and a beta subunit, gamma subunit or beta and gamma subunits tagged with a fluorescent or luminescent protein and a known receptor or an orphan receptor (a) in the absence of an added candidate molecule, (b) in the presence of an added molecule and then comparing the images of (b) with the images of (a) visually or by using appropriate image analysis computing software to determine whether images from (b) demonstrate translocation of the beta subunit, gamma subunit or beta and gamma subunits from the plasma membrane to the cell interior or translocation from cell interior to the plasma membrane of the cell.

A method of classifying candidate therapeutic molecules as agonists, antagonists or inverse agonists using biosensor cells encoding and expressing an alpha subunit and a fluorescent protein or luminescent protein tagged beta subunit, gamma subunit or beta and gamma subunits and screening for predicted changes in the images of these cells in response to the addition of the candidate molecules by direct visualization or using image processing software.

A method for identifying and classifying candidate therapeutic molecules which are agonists, antagonists or inverse agonists of various receptor types by performing high content screening of biosensor cells wherein ‘high content’ is defined as information about bisosensor activity in terms of both time dependence and spatial location in an intact cell maintaining structural and functional integrity.

### BRIEF DESCRIPTION OF THE DRAWINGS

FIG. 1 shows one method of operation of the biosensor cell.

FIG. 2 shows another method of operating the biosensor cell.

FIG. 3 shows images acquired using the imaging set up described in FIG. 1 of Chinese Hamster Ovary (CHO) cells expressing the M2 acetylcholine receptor, the G protein alpha-o subunit and the gamma11 subunit tagged with yellow fluorescent protein (YFP). The fluorescence emission from the gamma11 tagged fluorescent protein is captured. Before agonist addition the biosensor is localized to the plasma membrane. After agonist addition the biosensor translocates to the cell interior as shown. After the addition of the antagonist to the agonist treated cells the biosensor translocates back to the plasma membrane. The image shown after agonist addition was captured 180 seconds after the capture of the image before agonist addition. The image shown after antagonist addition was captured 80 seconds after the addition of the image after agonist addition.

FIG. 4 (left) shows the plot of emission intensities of YFP tagged to a gamma11 subunit type on the plasma membrane determined by using image processing program (Metamorph, Universal Imaging) and (right) shows a similar plot of the same YFP emission intensity from the same cells from the internal compartment. The cells were CHO cells expressing the M2 acetylcholine receptor and Galpha-o. Agonist was 100 μM carbachol and antagonist was 1 mM atropine.

In FIG. 5, the translocation of the YFP tagged gamma11 subunit is shown to be sensitive to the concentration of agonist used to activate the receptor in cells similar to those in FIG. 3.

FIG. 6 shows that translocation of YFP tagged gamma11 subunit is elicited by repeated applications of the agonist and antagonist to the same cell. Cells were as above in FIG. 3.

FIG. 7 shows that the YFP tagged gamma 11 subunit translocates in response to the activation of a distinctly different receptor, the 5HT1A serotonin receptor. Cells were CHO cells expressing introduced 5HT1A receptors, alpha-o, beta1 and YFP tagged gamma11.

FIG. 8 shows that the biosensor—YFP tagged gamma11 subunit translocates in response to the activation of an endogenous 5HT1B receptor. Cells were CHO cells expressing alpha-o, beta1 and YFP tagged gamma11.

FIG. 9 shows images of cells in which YFP tagged gamma subunit containing a different gamma subtype gamma 1 translocates in response to the activation of the M2 receptor in CHO cells expressing introduced alpha-o, beta1 and and gamma1.

FIG. 10 shows plots of the emission intensity of YFP tagged to gamma 1 that it translocates in response to the activation of the M2 receptor in CHO cells expressing introduced alpha-o, beta1 and gamma1.

FIG. 11 shows plots of the emission intensity of YFP tagged to gamma 5 indicating that it translocates in response to the activation of the M2 receptor in CHO cells expressing introduced alpha-o, beta1 and YFP tagged gamma5.

FIG. 12 shows plots of the emission intensity of YFP tagged to yet another gamma subtype, gamma 13 indicating that it translocates in response to the activation of the M2 receptor in CHO cells expressing introduced alpha-o, beta1 and YFP tagged gamma13.

FIG. 13 shows that a YFP tagged mutant gamma 11 subunit that is geranylgeranylated translocates in response to the activation of the M2 receptor in CHO cells expressing introduced alpha-o, beta1 and YFP tagged gamma11 mutant.

FIG. 14 shows that a YFP tagged mutant gamma 5 subunit in which the last 10 residues upstream of the C terminal Cys are deleted translocates in response to the activation of the M2 receptor in CHO cells expressing introduced alpha-o, beta1 and gamma deletion mutant.

FIG. 15 shows that a YFP tagged mutant gamma 5 subunit in which the last 10 residues upstream of the C terminal Cys are scrambled translocates in response to the activation of the M2 receptor in CHO cells expressing introduced alpha-o, beta1 and gamma scrambled mutant.

FIG. 16 shows images of cells in which a YFP tagged gamma 5 subunit which is mutated such that it is farnesylated translocates in response to the activation of the M2 receptor in CHO cells expressing introduced alpha-o subunit, beta1 and gamma farnesylated mutant.

FIG. 17 shows that a YFP tagged gamma 5 subunit which is mutated such that it is famesylated translocates in response to the activation of the M2 receptor in CHO cells expressing introduced alpha-o subunit, beta1 and gamma farnesylated mutant.

FIG. 18 shows that YFP tagged gamma11 translocates in response to the activation of a distinctly different class of muscarinic acetylcholine receptors—the M3 receptors—in CHO cells expressing introduced alpha-o-alpha-q chimeric subunit, beta1 and gamma11.

FIG. 19 shows that YFP tagged gamma11 translocates in response to the activation of yet another distinctly different class of receptors—the beta 2 adrenergic receptors—in CHO cells expressing introduced alpha-o-alpha-s chimeric subunit, beta1 and gamma11.

FIG. 20 shows that YFP tagged beta1 translocates from the plasma membrane when expressed with αo and gamma11 in response to an agonist and antagonist to M2 receptors

FIG. 21 shows images of cells in which translocation of YFP tagged G protein gamma11 in response to agonist or antagonist and resultant alteration in the pattern of fluorescence emission in the cell is stable for relatively long periods of time.

FIG. 22 shows the plot of emission intensity from YFP tagged to gamma11 from lung cells (HT1080) when the cells coexpressed αo-CFP, β1 and γ11-YFP with M2 and were exposed sequentially to agonist, carbachol and antagonist, atropine.

FIG. 23 shows the plot of emission intensity from YFP tagged to β1 from lung cells (HT1080) when the cells coexpressed αo-CFP and β1-YFP coexpressed with M2 and were exposed sequentially to agonist, carbachol and antagonist, atropine.

FIG. 24 is a diagrammatic representation of the translocation process in response to an agonist.

FIG. 25 is a diagrammatic representation of the subsequent translocation process in response to an antagonist when antagonist treatment follows agonist treatment.

### DETAILED DESCRIPTION OF THE INVENTION

This invention provides a functional intact biosensor cell comprising mammalian G protein subunits tagged to a fluorescent protein —mutants of GFP (Qreen fluorescent protein)—CFP (Cyan fluorescent protein) or YFP (Yellow fluorescent protein) that provide a detectable and discernible fluorescence signal. When expressed in a mammalian cell line and endogenous or introduced/added (expressed) receptors coupled to the G protein biosensors are activated, the beta subunit or gamma subunit or beta and gamma subunits translocates from the plasma membrane to the cell interior and subsequently when the biosensor cells are exposed to an antagonist the beta subunit or gamma subunit or beta and gamma subunits translocates from the internal region to the plasma membrane. Thus the images of the biosensor cell provide a direct quantitative, reproducible measure of the activity of a G protein coupled receptor.

In an aspect, a live functional G protein “biosensor cell” comprises a translocatable G protein beta or translocatable gamma subunit or translocatable beta and gamma subunits tagged with a fluorescent protein or a luminescent protein.

In an aspect, a live functional G protein “biosensor cell” comprises an endogenous or introduced G protein alpha subunit and introduced translocatable beta and gamma subunits one of which or both of which are tagged with a fluorescent or luminescent protein.

In an aspect, a live functional G protein “biosensor cell” comprises an endogenous or introduced G protein alpha subunit and an introduced translocatable gamma subunit tagged with a fluorescent or luminescent protein with an endogenous translocatable beta subunit.

In an aspect, a live functional G protein “biosensor cell” comprises an endogenous or introduced G protein alpha subunit and an introduced translocatable gamma subunit tagged with fluorescent protein and endogenous or introduced translocatable beta subunit.

In an aspect, a live functional G protein “biosensor cell” comprises an endogenous or introduced G protein alpha subunit and an introduced translocatable beta subunit tagged with fluorescent protein and an endogenous or introduced translocatable gamma subunit.

As used herein, the term “transformation or transfection” includes a process whereby a DNA construct (also called a vector, vector construct or plasmid) carrying foreign (referred to as a heterologous gene) is introduced into and accepted by a suitable host cell. Multiple genes may be operably linked in a single DNA construct and in another aspect multiple genes are introduced using separate vectors. In an aspect, the host cell having the stable DNA construct is cultured to create progeny biosensor cells.

Accordingly in an aspect, a DNA construct (or genetic construct) used for the expression of the biosensor in a suitable host cell such as Chinese Hamster ovary cells or progeny thereof comprises (a) a nucleotide sequence from a suitable cloning vector which capably allows for replication in a mammalian cell such as CHO, (b) regulatory sequences that are capable of allowing transcription and translation of the introduced G protein subunit genes (cDNAs) in CHO cells with or without tagged CFP and YFP, (c) a gene specifying a selectable marker that allows for the selection of cells containing stably integrated vector, and (d) similar construct containing a gene (CDNA) for a mammalian G protein coupled receptor.

In an aspect, the DNA or genetic construct further comprises an expression control sequence operably linked to a sequence encoding (and expressing) the expression product.

As used herein, the terms “DNA construct” or “genetic gene construct”, “gene” or “cDNA” are used interchangeably herein to, refer to a nucleic acid molecule which may be one or more of the following: regulatory regions, e.g. promoter and enhancer sequences (that are competent to initiate and otherwise regulate the expression of a gene product(s)); any other mutually desired compatible DNA elements for controlling the expression and/or stability of the associated gene product(s) such as polyadenylation sequences; other DNA sequences which function to promote integration of operably linked DNA sequences into the genome of the host cell and any associated DNA elements contained in any nucleic acid system (e.g. plasmid expression vectors) used for the propagation, selection, manipulation and/or transfer of recombinant nucleic acid sequences, sequences encoding proteins that are part of the biosensor or proteins that are functional G protein coupled receptors.

As used herein, the terms “regulatory DNA sequences” or “regulatory regions” or “DNA sequences which regulate the expression of” are used interchangeably herein refer to nucleic acid molecules which function as promoters, enhancers, insulators, silencers and/or other similarly defined sequences which control the spatial and temporal expression of operably linked and/or associated gene products.

In an aspect, the biosensor cell is contained in a suitable housing or compartment which includes multi well plates and imaging chambers wherein the cell will be either bathed, incubated or exposed to suitable liquid composition flow. In an aspect, the bathing or incubating liquid of defined composition may be added using appropriate fluid delivery systems that may be manually operated or operated robotically. In an aspect an imaging chamber liquid may flow through the chamber and through an exit, i.e. outflows through an opposite side. In an aspect, temperature controlling devices may be employed to control the temperature of incubating, bathing or flowing liquid.

Typically, the composition of the bathing, incubating or flowing liquid comprises Hank's buffered saline with 10 mM Hepes pH 7.4 and 1 mg/ml glucose Hank's Balanced Salt Solution (“HBSS”) and is prepared externally and introduced into the wells or compartments containing the biosensor cells or the imaging chamber manually or automatically using fluid delivery systems. HBSS is available from Hyclone, 1725 Hyclone Road, Logan, Utah 84321, U.S.A.

In an aspect, in the case of an imaging chamber the inflow composition flow rate is controlled so that the flow rate is about 1 m/min.

In an aspect, the outflow composition is collected from the biosensor cell via outlet manifold or connection and in an aspect, is vacuum aspirated. Flows are controlled by means of suitable valves such as a manual value or an automatic value.

Typically on starting up the biosensor cell and placing it on line i.e. in service, the cell is exposed to HBSS and the cells are brought into the focus of the objective of the microscope. A user selects the image timed exposure and starts to acquire images at the emission wavelength of the fluorescent protein tagged to the gamma or beta subunit by exciting the protein at an appropriate wavelength. In an aspect, the excitation and emission wavelengths are controlled by using filter wheels or an image splitting device. In an aspect, image acquisition is performed by a digital CCD camera which this is controlled by a software program on a computer such as a personal computer equipped with an operating system and a memory. In an aspect, components of the imaging chamber including inlet and outlet flow connections, valves, etc. are suitably operably connected and suitably functionally assembled and connected electrically (powered up and the electricity turned on), such as connected to a 110 volt electric supply so that the imaging chamber and biosensor cell performs in the intended way and function. In an aspect, the valves are manual or are electronically operated by an actuator mechanism under human or computer control.

The term “endogenous receptor” refers to an aspect where suitable G protein coupled receptors are present in a host cell and as such, an exogenous gene capably encoding and expressing a G protein coupled receptor is not necessary in any DNA construct for transcription and translation in cells due to the already present G protein coupled receptors.

The terms “introduced receptors” refers to an aspect where G protein coupled receptors are functionally encoded and expressed in a host cell such as by use of a suitable DNA construct competently integrated into the genome of the host cell, or transiently transfected such that the protein is expressed but the encoding DNA is not integrated in the genome, the construct comprising a nucleic acid encoding and expressing G protein coupled receptors.

As used herein the term “G protein” includes guanine nucleotide binding heterotrimeric proteins comprising alpha subunits, and translocatable beta subunits and translocatable gamma subunits that are stimulated by G protein coupled receptors resulting in the alpha subunit binding nucleotide GTP in place of nucleotide GDP and the beta or gamma or both beta and gamma subunits translocating.

As used herein the terms “translocatable or translocates or translocation or translocating or translocated” refer to the movement of the fluorescent protein tagged gamma subunit or beta subunit or the beta and gamma subunits from the plasma membrane of the cell to the cell interior as a result of the activation of specific receptors in the cell.

As used herein the terms “translocatable or translocates or translocation or translocating or translocated” refer to the movement of the fluorescent protein tagged gamma subunit or beta subunit or the beta and gamma subunits from the cell interior to the plasma membrane as a result of the inactivation of specific receptors in the cell.

As used herein the terms “translocatable or translocates or translocation or translocating or translocated” refer to the movement of the fluorescent protein tagged gamma subunit or beta subunit or the beta and gamma subunits from the plasma membrane of the cell to the cell interior or the movement from the cell interior to the plasma membrane as a result of the activation or inactivation of specific receptors in the cell.

As used herein, the term “functional” means that a biosensor cell operates, is fully operational in all its aspects and is capable of biosensor translocation in the biosensor cell.

In an aspect, the fluorescence signal from the biosensor molecule is expressed directly as the emission of YFP or CFP or any other fluorescent protein attached to the gamma or beta subunit or both subunits when that fluorescent protein is excited at an appropriate wavelength of light.

In an aspect, a functional biosensor produces a discernible, detectable and measurable fluorescence signal (or luminescence signal), an image (of captured fluorescence) which is competently reliably and accurately captured by visual inspection aided by a microscope or acquired by appropriate camera and computer software to be displayed visually on a computer monitor for a person for viewing. The intensity and duration of the fluorescence signal is detectable and is reproducible. The images of cells may be projected on a monitor and compared to another image of the cell after treatment with a full or partial agonistic, antagonistic or inverse agonistic, allosteric regulatory or innocuous compound on a monitor. A person can then visually compare such images and make a determination on whether there is a difference between the images compared. (Herein the alphabetical letters a, b, c, d, e, etc., are used to denote image characteristics attained from an operational biosensor cell.)

As used herein the term “fluorescent protein” refers to any protein that is genetically encoded and expressed as a fusion with a wild type or mutant G protein subunit type such that it emits a fluorescent signal that is detectable using appropriate methods when excited at the necessary wavelength of light.

As used herein, the term “GFP” refers to the Green Fluorescent Protein from Aequorea victoria [7].

As used herein, the term “CFP” refers to mutant forms of GFP that possess the fluorescence excitation and emission properties similar to the Cyan Fluorescent Protein [7].

As used herein the term “YFP” refers to mutant forms of GFP that possess the fluorescence excitation and emission properties similar to the Yellow Fluorescent Protein including second generation and third generation YFP mutants including Citrine and Venus [7].

In an aspect, useful nonlimiting illustrative fluorescent proteins include modified green fluorescent proteins including but not limited to those disclosed in U.S. Pat. No. 6,319,669 which issued to Roger Tsien on Nov. 20, 2001, Wavelength Engineering Fluorescent Proteins, Modified Green Fluorescent Proteins as disclosed in U.S. Pat. No. 5,625,048 which issued to Roger Tsien on Apr. 29, 1997 and Modified Green Fluorescent Proteins as disclosed in U.S. Pat. No. 5,777,079 which issued to Roger Tsien on Jul. 7, 1998.

As used herein the term “candidate drug molecule” includes at least one of a molecule, ion and chemical moiety for which it is desired to be identified and classified as having potential therapeutic value. The term “molecule” includes a single molecule as well as pools, collections, libraries and assemblies of several different molecules, cells and ions.

As used herein, the term “G protein coupled receptors” include proteins that sense a stimulus signal on one portion of the receptor and communicate it to another portion of the receptor that acts on a heterotrimeric G protein(s). Illustratively non-limiting stimulus signals range from but are not limited to one or more of neurotransmitters, hormones, synthetic and natural agonists, light, odorant and gustatory molecules.

Illustrative useful non-limiting mammalian G-protein coupled receptors include Class A Rhodopsin like; Class B Secretin like; Class C Metabotropic glutamate (see http://www.gpcr.org/7tm/).

Characterized or uncharacterized (orphan) receptors include those that are capable of activating G proteins in response to a stimulus. These are also included as G protein coupled receptors.

As used herein, the term “de-orphaning” includes a method of discovering/identifying a molecule as binding to an orphan receptor or likely binding to an orphan receptor and eliciting predicted images from the G protein cell biosensor. With the identification of a molecule which binds to an orphan receptor, the orphan receptor is de-orphaned. Genomics and proteomics initiatives of human and other mammals have yielded a vast reservoir of information about the nucleic acid and amino acid sequences of potential G protein coupled receptors without yielding direct information about the stimulus signal including but not limited to natural or synthetic molecules that activate the receptor and the G protein that couples to the receptor. Genomic and proteomic information can indicate that some of these uncharacterized orphan receptors may be at the basis of disease. De-orphaning i.e. identifying the molecules that bind to these receptors, thus is of direct immense therapeutic utility in disease causation studies and diagnosis.

As used herein the term “ligand” includes hormones, neurotransmitters and other natural or synthetic chemical molecules, including ions and chemical moieties that have the capability to specifically and effectively bind to a G protein coupled receptor so as to produce an activated G protein or antagonize such activity initiated by another ligand.

G proteins comprising alpha, beta and gamma subunits may be considered as in their respective resting state when bound to GDP. A G protein coupled receptor that is stimulated by a chemical or physical stimulus activates a G protein capable of coupling with it and replaces the GDP with GTP and the G protein is activated. Without being bound by theory, the alpha subunit is thought to dissociate from the betagamma complex. The hydrolysis of the GTP by the GTPase activity of the alpha subunit result is thought to deactivate the alpha subunit and its reassociation with the betgamma complex resulting in a return to the resting state.

As used herein the term “activated G protein heterotrimer” refers to the activation of the G protein alpha subunit wherein the G protein alpha of subunit binds GTP giving up GDP and undergoes a conformational change.

Without being bound by theory, it is believed that in the native state a hormone or neurotransmitter molecule binds to a G protein coupled receptor outside the cell and stimulates a change in the G protein coupled receptor that allows the receptor to activate a G protein capable of coupling to the receptor.

The G protein subunits activated in this fashion regulate the activity of various effectors inside the cell that bring about changes in cellular physiology.

As used herein, the term “effector” includes a molecule or chemical moiety which is an intracellular target of G protein alpha subunit and betagamma complex. Illustratively, nonlimiting major effectors include adenylyl cyclase, phospholipase C and ion channels among others which regulate the levels of second messengers such as cAMP, IP3 as well as ions.

Extracellular signals are sensed by a biosensor cell and transduced into intracellular regulatory changes which result in the final physiological response to the initial stimulus. The intrinsic ability of activated G protein subunits to deactivate is accelerated by a large family of regulatory proteins in mammalian systems. The activated subunits thus go back to the resting state allowing a G protein to act as a molecular switch that is in an “on” or “off” state reflecting the stimulated or unstimulated state of the receptor.

As used herein, the term “agonist” refers to and includes any natural or synthetic molecule, ion or chemical moiety that is capable of stimulating a G protein couple receptor such that a G protein capable of coupling with that receptor is activated.

As used herein, the term “antagonist” refers to and includes any natural or synthetic molecule, ion or chemical moiety that is capable of inhibiting the action of an agonist by interacting directly or indirectly with the receptor.

As used herein, the term “inverse agonist” refers to and includes any natural or synthetic molecule, ion or chemical moiety that is capable of increasing the proportion of inactive receptors in a receptor population comprising active and inactive receptors by binding with higher affinity to the inactive receptors in comparison to its binding with the active receptors [8].

As used herein, the term “allosteric regulator” refers to and includes any natural or synthetic molecule, ion or chemical moiety that is capable of interaction with a receptor at a site other than the site that normally binds its native ligand but nevertheless alters the function of the receptor.

As used herein, the term “innocuous” refers to and includes any natural or synthetic molecule, ion or chemical moiety that is not capable of any measurable effect on the receptor function.

Without being bound by theory, it is believed that in the G protein biosensor cell herein, the fluorescent protein tagged gamma subunit, or beta subunit occur as a complex with the alpha subunit that maybe introduced or endogenous to form a heterotrimer that is activated by the receptor resulting in the translocation of the gamma subunit and the beta subunit from plasma membrane to internal region and then sue to subsequent exposure to an antagonist translocation back to the plasma membrane.

Illustrative useful living non-limiting competent host mammalian cells include but are not limited to Chinese Hamster ovary cells, Human Embryonic Kidney Cells, COS cells, NIH 3T3 cells, HEK 293 cells, and Swiss 3T3 cells.

Illustrative useful living non-limiting competent host mammalian cells include but are not limited to differentiated cells such as cardiomyocytes, neurons and cells from various mammalian tissues.

Illustrative useful living non-limiting competent host cells include other metazoan cells such as Sf9 insect cells, avian QT6 cells and Drosophila Schneider cells.

Useful non-limiting compounds and molecules which may be added to a biosensor cell for evaluation as a therapeutic candidate include but are not limited to those candidates which are available in libraries of candidate therapeutic drug molecules from industrial, commercial and research laboratory sources.

As used herein “image” refers to the image of a cell in which the spatial distribution of the fluorescent biosensor molecule can be discriminated to an extent where its presence on the plasma membrane an internal regions of the cells can be distinguished sufficiently to determine whether translocation of the biosensor from one region to another has occurred.

In an aspect, a method for determining signal transduction activity in a live functional cell (system) using image analysis comprises (reactively) exposing a biosensor cell comprising a G protein coupled receptor and fluorescent protein tagged G protein gamma or beta or both subunits to potential full or partial agonists, antagonists, inverse agonists and allosteric regulators and quantifiably measuring G protein receptor signaling activity non-invasively in the intact cell by measuring the extent of translocation of the beta, gamma or both subunits.

In an aspect, a non-invasive screening method for identifying agonist candidate therapeutic drug molecules comprises using an intact live biosensor cell system that contains a receptor and G protein biosensor which when exposed to a candidate therapeutic molecule results in the translocation of the biosensor to the internal region from the plasma membrane indicating that said candidate is an agonist therapeutic drug molecule.

In an aspect, a non-invasive screening method for identifying natural or chemically synthesized candidate agonists, antagonists, inverse agonists and allosteric regulators that bind to uncharacterized or “orphan” mammalian receptors thus de-orphaning orphan receptors comprises using an intact living biosensor cell containing said orphan receptor and exposing to a candidate therapeutic molecule to obtain images before and after the addition of molecules and based on the comparison of images identify agonists as those that induce translocation of the biosensor to an internal region from the plasma membrane and antagonists as those that induce translocation of the biosensor to the plasma membrane from the internal region thus de-orphaning the receptor.

In an aspect, a classification method for natural or chemically synthesized candidate agonists, antagonists and inverse agonist that bind to previously characterized, uncharacterized or orphan receptors, comprises operating an intact living insect cell where the G protein biosensor comprising the fluorescent protein tagged beta, gamma or both beta and gamma subunits are expressed using a baculovirus vector along with the alpha subunit and obtaining images in the presence or absence of candidate therapeutic molecules and comparing these images to identify agonists, antagonists, inverse agonists and allosteric regulators for the receptors.

In an aspect, a method for increasing receptor types that will couple to the functional biosensor comprising G protein beta, gamma or both beta and gamma subunits fused to fluorescent protein by mutationally altering the C terminal tail of the alpha subunit constituent of the biosensor.

In an aspect, a method for altering the intensity of the G protein beta, gamma subunit translocation response by mutationally altering the intrinsic biochemical properties of the alpha subunit or beta subunit or gamma subunit or combinations of these subunits that constitute the biosensor.

In an aspect, a method for altering the intensity of the the gamma or beta subunit or beta and gamma subunits translocation in response to agonist, antagonist, inverse agonist and allosteric regulator molecules comprises mutationally introducing pertussis toxin insensitivity into the functional biosensor comprising G protein signaling subunits.

In an aspect, a method for identifying a candidate therapeutic drug molecule is provided which comprises obtaining images of a live functional biosensor cell comprising a G protein alpha subunit and a fluorescent protein tagged gamma or beta or both beta and gamma subunits and also containing a previously defined receptor or an orphan receptor (a) in the absence of an added candidate molecule, obtaining images of said biosensor over a time period, (b) in the presence of an added molecule and comparing said images (b) with said images (a) to obtain a comparison of images of (b) and (a).

If the comparison shows that translocation of the fluorescent tagged gamma subunit or beta subunit or both beta and gamma subunits after the addition of a candidate molecule from the plasma membrane to an internal region (b) compared to the images before the addition of the candidate (a), then one classifies the molecule as an agonist candidate therapeutic drug molecule. If the images (b) are similar to said images (a), then one classifies the molecule as a molecule likely innocuous not having agonistic therapeutic value.

As used herein the term “classifies” includes making a determination and assessing the priority of as regards continued and/or future testing and evaluation of a candidate molecule for therapeutic efficacy of a candidate molecule in the development of remedial and preventative and better medicines for humans and other primates. Illustratively, the comparison is visual by visually comparing images with another or by using automated systems using appropriate image processing/pattern recognition software image acquiring devices.

In an aspect, a classification includes a determination that a molecule is to advance, remain or be removed from testing, be advanced in testing, keep its placement in testing in research or development. In an aspect, a classification includes a determination that a molecule is not to be further tested, i.e., testing in that molecule is to be terminated. In an aspect, a classification includes a ranking or prioritization of work, such as further work to be done or not to be done on the molecule.

In an aspect, a number of different molecules are added to the biosensor singly or as a pool of various candidates. Independent images of biosensor cells after and before the addition of these candidate molecules are obtained.

In an aspect, a method further comprises adding to the biosensor cells, a molecule known as an agonist to provide images (c) from the biosensor cells and subsequently adding to said biosensor cells a candidate therapeutic drug molecule to obtain images (d) and then compare the images (d) with images (c). The images resulting from exposure to the known agonist establishes a baseline image set of the biosensor cell for use in other comparisons using the novel methodology and biosensor herein.

If the images from the biosensor cell after the addition of a candidate molecule in (d) shows translocation of the fluorescent tagged gamma or beta or both beta and gamma subunits from the internal region to the plasma membrane compared to images (c), then one classifies the molecule added second as an antagonist therapeutic drug molecule.

If the images from the biosensor cell after the addition of a candidate molecule in (d) shows no change in spatial distribution of the fluorescent tagged gamma or beta or both beta and gamma subunits compared to images (c), then one classifies the molecule added second as an innocuous.

In an aspect, a method is provided for identifying a therapeutic drug molecule as an inverse agonist which comprises obtaining images (e) from biosensor cells containing overexpressed or mutant receptors of known (characterized), or orphan status possessing constitutive receptor activity such that the images (e) of the said biosensor cells indicate translocation of the fluorescent tagged gamma or beta or both subunits to the plasma membrane from the internal region compared to images (a) from the biosensor cells before addition of any molecule.

If addition of the candidate does not significantly alter the images (e), then the added molecule is classified as innocuous.

In an aspect, comparison of the respective images provides the capability of determining whether the candidate molecule is classified as an agonist, an antagonist, an inverse agonist or as innocuous.

In an aspect, a method is provided for identifying a therapeutic drug molecule as an allosteric regulator of a receptor which comprises obtaining images (f) from biosensor cells containing known (characterized) or orphan receptors in the presence of a known agonist or antagonist or inverse agonist and comparing said images (f) with images of biosensor cells exposed to the agonist or antagonist or inverse agonist (g) of the said biosensor cells to determine whether translocation of the fluorescent tagged gamma or beta or both subunits from one region of the cell to another region is altered in (i) compared to (g) and if altered classify the molecule as an allosteric regulator of that receptor.

In an aspect, a non-invasive method is provided for classifying therapeutic candidate molecules, where the mammalian G protein biosensor molecules are expressed in insect cells using a baculovirus vector for classifying candidate therapeutic drug molecules by obtaining images and comparing them in a manner recited above.

If desired receptor types that will couple to the biosensor are altered by mutationally altering the C terminal tail of the alpha subunit constituent of the biosensor directing the biosensor to couple to and elicit changes in the spatial cellular distribution of the fluorescent signal from receptors that do not normally couple to that biosensor.

In an aspect, a method is provided for eliciting changes in the spatial cellular distribution of the fluorescent signal from biosensors that are not normally responsive to a receptor by mutationally altering the intrinsic biochemical properties of the subunits that constitute the biosensor such that changes in the spatial cellular distribution of the fluorescent signal is elicited on activation of the mutant biosensor by a receptor.

In an aspect, a method is provided for altering the intensity of the response seen in the images to agonist, antagonist, inverse agonist and allosteric molecules by mutationally introducing pertussis toxin insensitivity into the biosensor and/or reducing the concentration of endogenous G protein subunits in cells containing the biosensor cell.

While the term “changes in the spatial cellular distribution of the fluorescent signal or translocation of the fluorescent protein inside the cell” have been used in this specification, claims and examples, the terms including “translocation of fluorescent protein” are intended to include emission spectra that are capably measured by any appropriate measurement methodology including but not limited to imaging using image scanners that analyze multiple individual cells for changes in the spatial distribution of fluorescence signal detection such as Kineticscan and Arrayscan from Cellomics, a fluorescence microscope with suitable optical filters or image splitter, CCD camera (illustratively a charged coupled device), computer and appropriate computer useful software, spectroscopy such as a fluorometer.

In an aspect, a useful imaging system comprises integrated or non-integrated systems containing devices for detecting the images of one or many individual cells, the fluorescence emission pattern at the subcellular level, software to analyze these changes and identify the appropriate cells in which changes have occurred and those in which such changes have not occurred such as but not limited to high throughput image readers like Arraycan reader and Kineticscan reader from Cellomics. In an aspect an imaging system includes a Zeiss Axioscope/Axiovert or Nikon Eclipse fluorescence microscope, filters from Chroma or Omega, CCD cameras from Hamamatsu or Roper and software from Metamorph from Universal Imaging or IP Lab from Scanalytics and a sufficiently powerful computer capable of running the appropriate software.

In an aspect, a biosensor cell comprising a mammalian G protein alpha subunit is tethered to the C terminus of a G protein coupled receptor through its N terminus and the beta or gamma or both beta and gamma subunits tagged with a fluorescent protein to provide detectable and discernible changes in the spatial cellular distribution of the fluorescent signal. When expressed in a mammalian cell line and the receptor is stimulated with an agonist, the changes in the spatial cellular distribution of the fluorescent signal are detected. Thus the changes in the spatial cellular distribution of the fluorescent signal from the biosensor cell provide a direct quantitative and reproducible measure of the activity of a G protein coupled receptor.

### General Procedure for Designing and Operating a Functional Biosensor Cell Providing Emission Spectra to Classify Candidate Molecules

Materials: Except listed, all chemicals were from Sigma Aldrich, St. Louis, Mo. Cells were grown in CHO IIIa medium (Life Technologies, 2575 University Ave., St. Paul, MN 55113) supplemented with charcoal stripped (CHO-Seratonin) or dialyzed fetal bovine serum (CHO-M2, CHO-M3, ACHO-B2-Adrenergic cells—Atlanta Biologicals, Atlanta, Ga.), glutamine, fungizone, penicillin/streptomycin and/or methoxetrate.

Suitable DNA constructs were designed and made as follows.

All were transferred to mammalian expression vectors pcDNA3.1 or pDEST12.2. The number of M2 receptors expressed was about 400,000 receptors per cell. CHO cells expressing M2, M3, B2-Adrenergic or 5-HT receptors were transfected with alpha alpha-o, alpha-o-CFP, alpha-o-alpha-q-CFP, alpha-o-alpha-s-CFP, beta1, YFP-gamma5, YFP-gamma1, YFP-gamma11, CFP-gamma11, YFP-gamma13, YFP-gamma5-farnesylated mutant, YFP-gamma11 -geranygeranylated mutant using Lipofectamine 2000 (Life Technologies, 2575 University Ave., St. Paul, Minn. 55113).

In these examples, multiple genes were introduced into a host cell (CHO) by means of separate DNA constructs by co-transfection.

Typically the inflow contains Hank's buffered saline with 10 mM Hepes pH 7.4 and 1 mg/ml glucose (HBSS) and is prepared external to the imaging chamber and introduced into the chamber manually by injection or using an automated electronic valve controlled system.

In an aspect, the inflow flow rate is controlled so that it is about 1 m/min.

In an aspect, the inflow composition to the imaging chamber is provided to the imaging chamber by means of a suitable connection thereto such as a manifold or a single or multi-port inlet.

Typically on starting up the biosensor cell it is exposed to HBSS, the cells are brought into the focus of image detection system. CC, CY and YY images are acquired at defined exposure times at defined intervals. In an aspect, image acquisition is controlled by appropriate image processing software operating on a computer or by visually scanning the images.

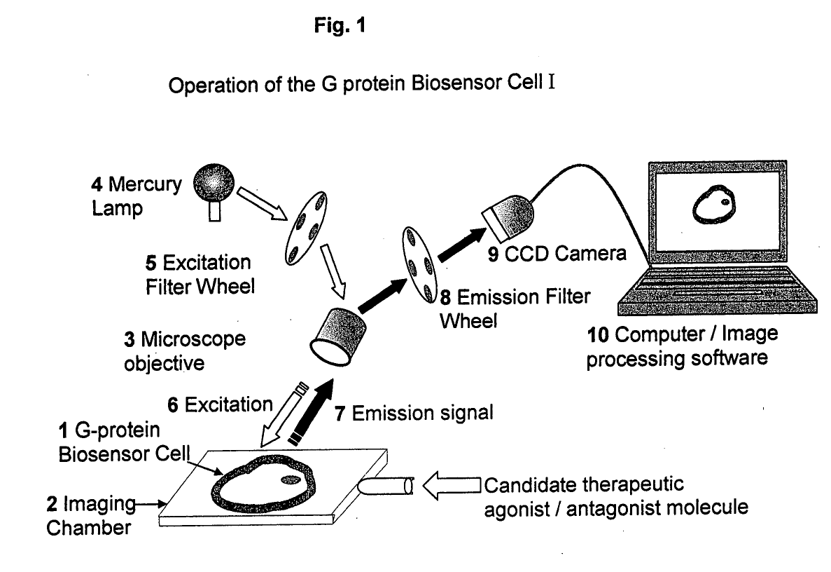

Image acquisition and analysis (i.e. image capture, recording and analysis) were carried out as follows (generally following the illustration in FIG. 1). Cells were seeded on glass coverslips (22×40 mm #2 from Fisher Scientific) in 60 mm dishes and cultured overnight for imaging. Coverslips containing cells were mounted in an imaging chamber of 25 μl internal volume (RC-30 from Warner Instrument Corporation, 1141 Dixwell Ave., Hamden, Conn. 06514) containing Hank's Buffered Saline Solution (HBSS) supplemented with 10 mM Hepes pH 7.4 and 1 mg glucose/ml. The imaging chamber was stage-mounted in an upright Zeiss Axioscope fluorescence microscope. Cells were observed with a Zeiss 63×(1.4 NA) objective.

Agonists, antagonists or other molecules in the HBSS solution were injected manually (or using an automated valve based system driven pneumatically or by gravity) at a rate of about 1 ml/min for 2-3 min through an inlet in the imaging chamber. Images were acquired at the indicated times. Cells were illuminated with a 100 W mercury lamp through a 3 or 10% neutral density filter. The filter wheels were run by a Sutter Lambda 10-2 device, a high speed excitation filter wheel that utilizes a direct stepper motor. (Sutter Instrument Company, 51 Digital Drive, Novato, Calif. 94949). In an aspect, power to operate instruments such as the microscope, pumps, motor(s), camera(s), computer(s) control system is supplied by 110 volt electricity which is supplied to the instruments and turned on at the startup.

Filters were used in combination with appropriate beam splitters in the filter cube. For CC (cyan) images: D430/25 excitation (x), D470/30 emission (m) and 455DCLP beam splitter; for CY (fluorescence) images: D535/30m and 455DCLP beam splitter; for YY (yellow) images: the filters D500/20x, D535/30m and 515LP beam splitter. All filters were from Chroma. Images were acquired using a Hamamatsu CCD Orca-ER Camera with different levels of binning. Exposure times were 0.8 and 1.5 seconds for each CC or CY or YY image with 4×4 binning. Images were acquired every 20 sec for a total of 10 or more minutes and stored as 12-bit gray scale image-stacks using Metamorph software (Universal Imaging Corporation, 402 Boot Road, Downington, Pa. 19335). Both camera and filter wheels were controlled peripherally using Metamorph from a Dell Computer Workstation (Dell Computer, Houston, Tex.) Images were processed using Metamorph (Universal Imaging) in a Dell Computer Workstation. Images were background subtracted, aligned and plasma membrane regions of entire cells (or most of the cell) were selected after determining that CC (wild type or mutant a subunit-CFP) and YY (wild type or mutant gamma subunit-YFP or beta subunit-YFP) signals were co-localized and were of approximately equal intensities. In some cases cells emitting distinctly different intensities of CC and YY emission were selected to examine the effect of differential expression of the two subunits relative to each other on the biosensor translocation properties. Average intensities in these regions were measured. It was ensured that maximum intensity per pixel in selected regions was lower than the maximum value on the available 12-bit gray scale (4095).

M2 expressing CHO cells were stably transfected with DNA constructs expressing G protein subunits respectively fused to CFP and YFP. CFP is inserted after Gly92 of the alpha-o subunit. YFP is fused to the N terminus of the beta-1 subunit or the wild type or mutant gamma subunit types of gamma5, or the wild type or mutant gamma11, or gamma13 or gamma1.

The YFP molecule is fused to the N terminus of the beta subunit or the N terminus of the gamma subunit based on our model.

The CFP (molecule) was inserted downstream of Gly92 in alpha-o since this region forms a loop that projects away from the betagamma complex in the crystal structure of the G protein.

Mutants of gamma5 encoding DNA and gamma11 encoding DNA were made using polymerase chain reaction with oligonucleotide primers that changed the nucleic acid sequence in the parent molecule to the mutant molecule.

The gamma11 subunit with the amino acid sequence for geranylgeranylation was mutated such that the DNA encoded CSFL instead of CVIS at the C terminus.

The gamma5 subunit with the amino acid sequence for famesylation was mutated such that the DNA encoded CVIS instead of CSFL at the C terminus.

The gamma5 deletion subunit with 10 amino acids deleted upstream of the CAAX box was mutated such that the DNA sequence encoded TGVSS - - - CSFL instead of TGVSSSTNPFRPQKVC at the C terminus.

The gamma5 subunit was mutated such that the C terminal sequence was scrambled—TPVNFSQVSKCSFL instead of STNPFRPQKVCSFL in the case of the wild type.

The chimeric molecule made up of alpha-o subunit containing the C terminal eleven amino acids of alpha-q were made using oligonucleotide primers and polymerase chain reaction.

To make the alpha-o-alpha-q chimera the alpha-o protein was mutated such that DNA encoded the C terminal eleven amino acids of alpha-q (LQLNLKEYNLV) instead of that of alpha-o (IANNLRGCGLY).

CHO cells stably transfected with the M2 muscarinic receptor were used to transfect alpha-o-CFP, beta1-YFP and one of the wild type or mutant gamma subunit cDNAs. Biosensor cells expressing appropriate combinations of subunits were imaged as described. Appropriate excitation and emission filters were used to detect and measure emission spectra including CFP emission after CFP excitation (CC) and YFP emission with YFP excitation (YY).

FIG. 1 depicts in an aspect, an operational process of acquiring and capturing fluorescence images for processing from a non-invasive biosensor cell containing a G protein biosensor described in more detail hereinafter in the Detailed Description of the Invention.

As regards FIG. 1, illustratively, G protein biosensor cells (1) are seeded on a glass coverslip and cultured overnight for imaging. Coverslips containing biosensor cells (1) are mounted in an imaging chamber (2) containing appropriate bathing solution. The imaging chamber (2) is stage-mounted in a fluorescence microscope. Cells (1) are observed with a microscope objective (3) with high magnification and numerical aperture. G protein biosensor cell (1) is excited with appropriate wavelengths of light using a mercury lamp (4) and optical filters (5). The excitation (6) produces an emitted fluorescence from the functioning G protein biosensor cell (1) as a fluorescence signal (7) which is collected by microscope objective (4) and passed through appropriate emission spectra wheel filter (8) to record an image in a cooled CCD camera (9) (charge coupled device) which transfers the image to a computer (10). The acquired image is processed using appropriate functional image processing software. Regions on the cell membrane expressing the biosensor are selected from images collected serially over time and the intensity of the signal emissions of differing spectra under different excitation spectral conditions are determined. Candidate therapeutic agonists, antagonists, inverse agonists and allosteric molecules are introduced by manual injection or using an automated fluid delivery system containing electronically driven valves into imaging chamber (2) using an inlet. In an aspect, the electronically driven valves, filter wheels, microscope lamp, CCD camera and computer are powered by 110 volt electric power.

It is understood that main and auxiliary components illustrated in FIG. 1 and in the biosensor are communicative with one another in a manner providing for full functionality of the biosensor cell including all needed electrical supply (including charge coupled devices such as a camera) and liquid conveying means including manifold connections to/from connected tubing, piping etc.

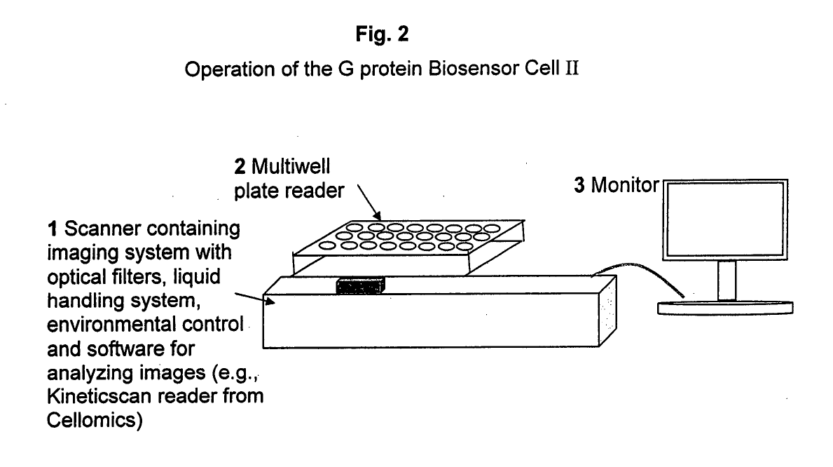

As regards FIG. 2, in an alternative method the biosensor cell can be operated by obtaining the single cell images from biosensor cells exposed to various candidate therapeutic molecules separately by using a scanner (1) that has the ability to obtain single cell images of sufficiently high resolution from the wells of multiple well plates (2) containing the cells and analyze the images before treatment of the cells with the molecule and after treatment of the cells with the molecules helping identify the cells that show changes in the distribution of the fluorescent or luminescent biosensor protein inside the cells. These changes can be viewed using a monitor (3). The scanner can include the requirements for imaging the cell, the liquid delivery system for introduction of cells as well as the candidate molecules, environmental control of cells and software for processing and analyzing single cell images for high throughput and high content screening.

The changes in the spatial cellular distribution of the fluorescent signal from the fluorescent protein tagged beta, gamma or both subunits are measured by comparing images of biosensor cells before and after exposure to a molecule that may activate or inactivate a receptor in the cell. The extent of translocation of the beta, gamma or betagamma subunits provides a direct measure of G protein activation over time.

Cell lines expressing fluorescent subunits showed direct, specific translocation of the beta-YFP, gamma-YFP or betagamma-YFP from plasma membrane to the cell interior of a cell in response to an agonist molecule when these proteins were co-expressed with the alpha subunit or with endogenous alpha subunits showing functional operation of the biosensor cell (i.e., it is activated.)

Cell lines expressing fluorescent subunits showed direct, specific translocation of the beta-YFP, gamma-YFP or betagamma-YFP from the cell interior to the plasma membrane in response to an agonist molecule when these proteins were co-expressed with the alpha subunit or with endogenous alpha subunits showing functional operation of the biosensor cell (i.e., it is activated.)

The results of our imaged functional biosensor cells show that alpha-CFP, beta-YFP or gamma-YFP or beta-YFP and gamma-CFP are localized predominantly in the biosensor cell plasma membrane. The distribution of alpha-CFP is similar to the distribution of the beta-YFP or gamma-YFP suggesting that the beta and gamma subunits are likely mostly present in the G protein heterotrimer form.

### EXAMPLES

Examples (1-19) following are provided to illustrate the invention and are not included for the purpose of limiting the invention in any way.

### Example 1

A mammalian G protein biosensor was prepared following the aforementioned procedure to express G protein beta1 subunit and gamma11 subunit tagged with YFP with the alpha-o subunit tagged with CFP.

The biosensor cell of this biological system is living because it has been cultured on the cover glass to which they are attached during operation and they have multiplied there and also respond to an extracellular signal with expected physiological response. The ability to reproduce and respond to the environment characterizes them as living.

The biosensor cell is intact because we have observed the biosensor cells before, during and after operating it and seen the cells under the microscope to retain their cytoplasmic contents within the plasma membrane.

The biosensor cells are functional because they respond to specific stimuli that act on particular receptors evoking anticipated responses.

The biosensor cells are in an appropriate state for starting the capture of signals from the cell when sequential images captured during imaging with a buffer indicate a stable fluorescence signal in the CC, CY and YY channels.

The stability of the base line signals emitted in the CC, CY and YY channels indicate that the environment of the biosensor cell (imaging chamber) and the cell are in a functional steady state.

### Example 2

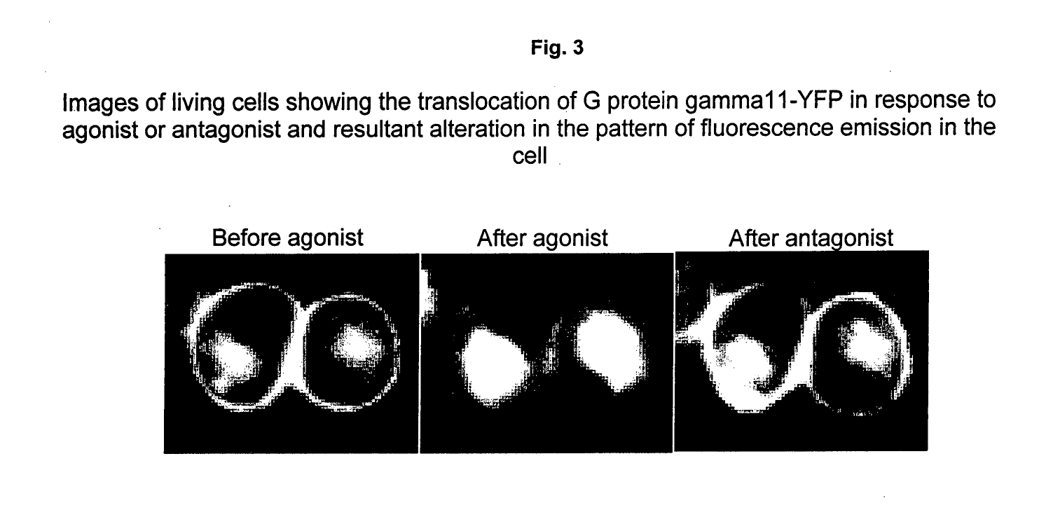

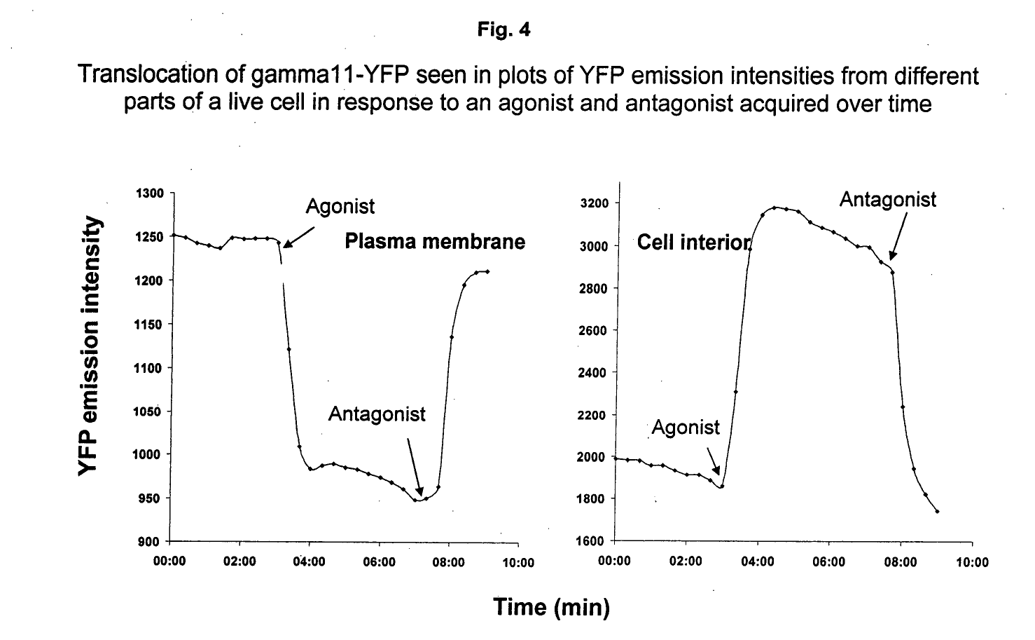

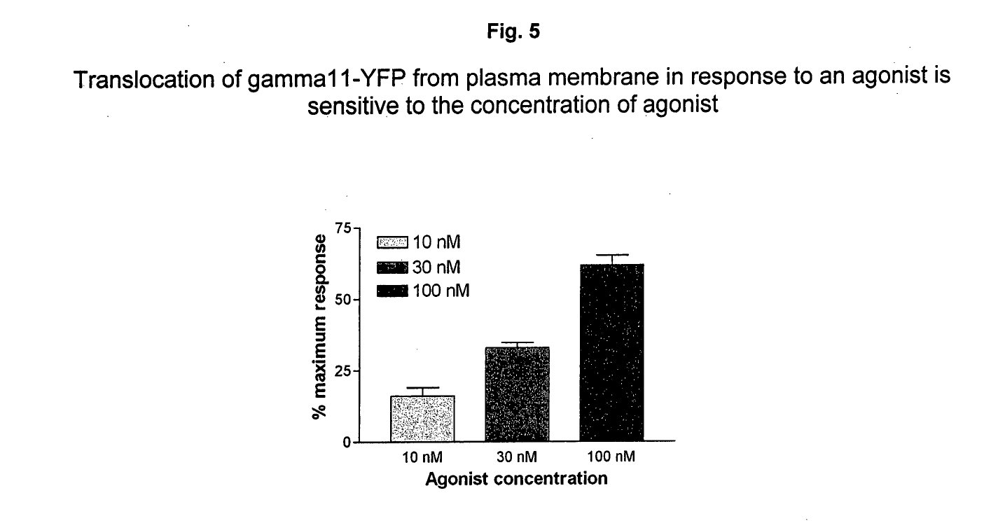

The functional G protein biosensor cell responded to an agonist molecule. Images of biosensor cells were acquired (captured) at regular time intervals before and after the addition of a muscarinic acetylcholine receptor agonist drug, carbachol. The images containing the fluorescent protein emission are shown in FIG. 3. The fluorescent signal intensity decreases on the plasma membrane and increases simultaneously inside the cell in the presence of the agonist molecule, carbachol. Subsequent addition of the antagonist molecule reverses this change, that is, the fluorescence intensity inside the cell decreases and the intensity on the membrane increases (FIG. 3). Fluorescent images of biosensor cells were acquired and analyzed before and after exposure to agonist and antagonist as described in the Procedures for Design and Operation following. The plots showing the change in fluorescence emission from the plasma membrane and the increase of emission inside the cell are shown in FIG. 4. Timing of agonist and antagonist additions to the biosensor cell are indicated with arrows on FIG. 4. Plots show a downward trend because of partial bleaching over the period of the test. The graph is representative of data from ten tests.

### Example 3

The functioning G protein biosensor cell responded quantitatively and reproducibly to an agonist molecule. The response of biosensor cells to the addition of varying concentrations of carbachol were measured as described earlier. The fluorescence signal intensity changes on the plasma membrane directly and is negatively correlated with increases in agonist drug concentration (FIG. 5). The fluorescence signal intensity changes inside the cell are directly and positively correlated with increases in agonist drug concentration. Points are means ±SEM of two tests. Tests were performed as described in Detailed Description of the Invention.

The EC50 for carbachol activation of the G protein is between 30-100 nM which is consistent with the EC50 for carbachol mediated M2 activation of a G protein measured in a reconstituted system.

### Example 4

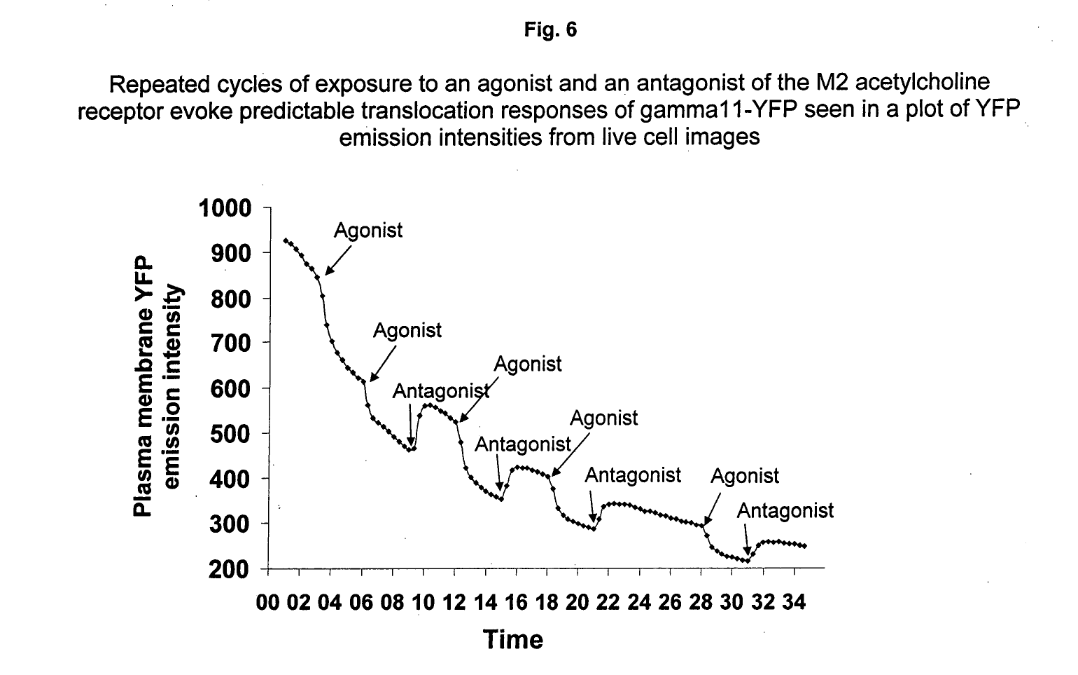

When biosensor cells were exposed sequentially to antagonist followed by agonist in repetitive cycles, the fluorescent signal translocated in a predictable manner repeatedly from the plasma membrane to the cell interior and then from the cell interior to the plasma membrane (FIG. 6).

### Example 5

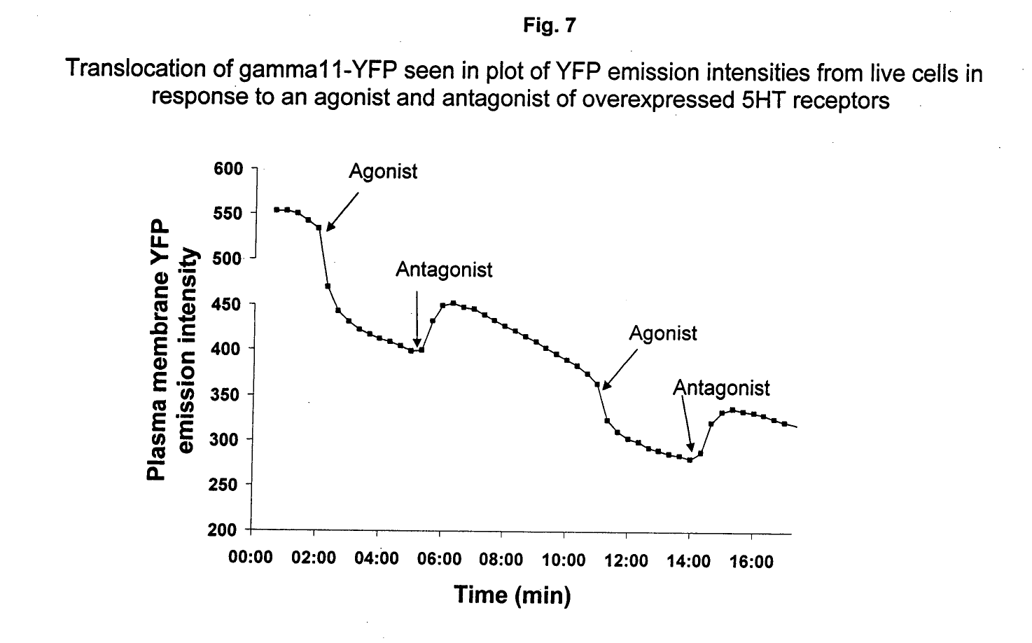

The biosensor cells expressing the serotonin receptor (5HT1A) respond to both an agonist (serotonin-5 hydroxytryptamine) and an antagonist (cyanopindolol) in a predictable fashion by showing translocation of the fluorescence signal from the YFP tagged gamma subunits from the plasma membrane to an the cell interior then from the cell interior to the plasma membrane (FIG. 7).

The response of the biosensor cells to serotonin establishes the ability of the biosensor cells to respond to the stimulation of more than one receptor type.

### Example 6

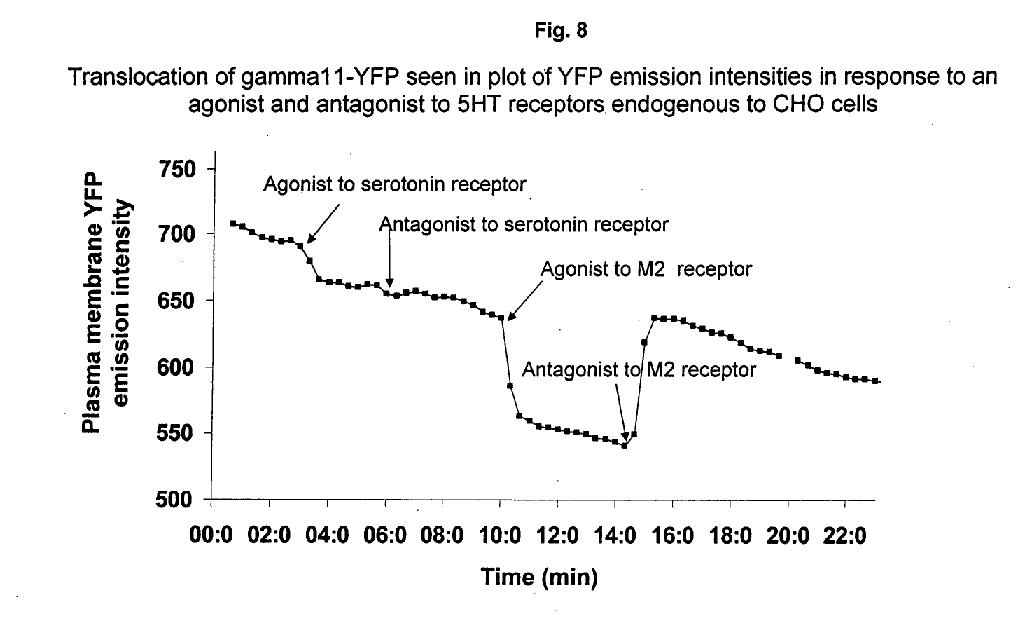

The biosensor cells respond in a predictable and previously established manner to the action of an agonist and an antagonist of a serotonin receptor (5HT1B) that is endogenous (not introduced or overexpressed) to CHO cells (FIG. 8).

The response of the biosensor cell to an endogenous receptor establishes the ability of the cell to respond predictably to endogenous as well as introduced receptors.

### Example 7

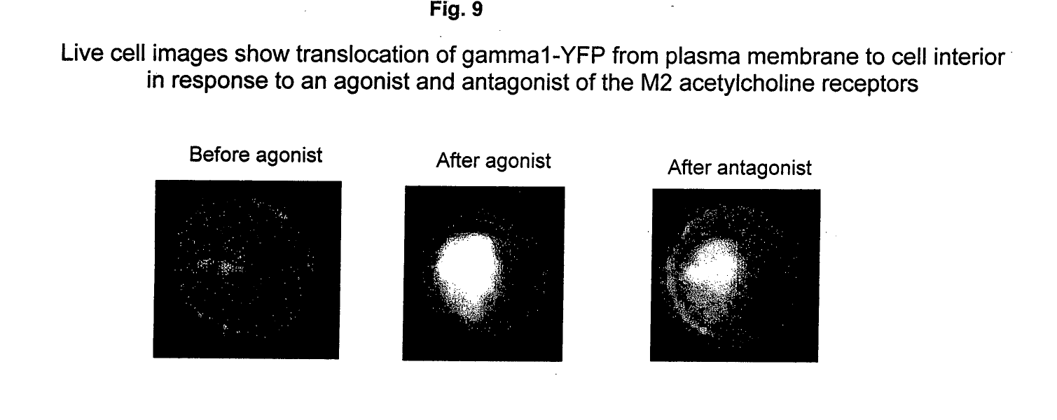

Single cell images of biosensor cells coexpressing a different gamma subunit type (gamma 1) tagged with YFP along with beta1 and alpha-o respond to the action of an agonist and an antagonist of the expressed M2 muscarinic receptors similar to biosensor cells expressing introduced gamma11 as previously established (FIG. 9).

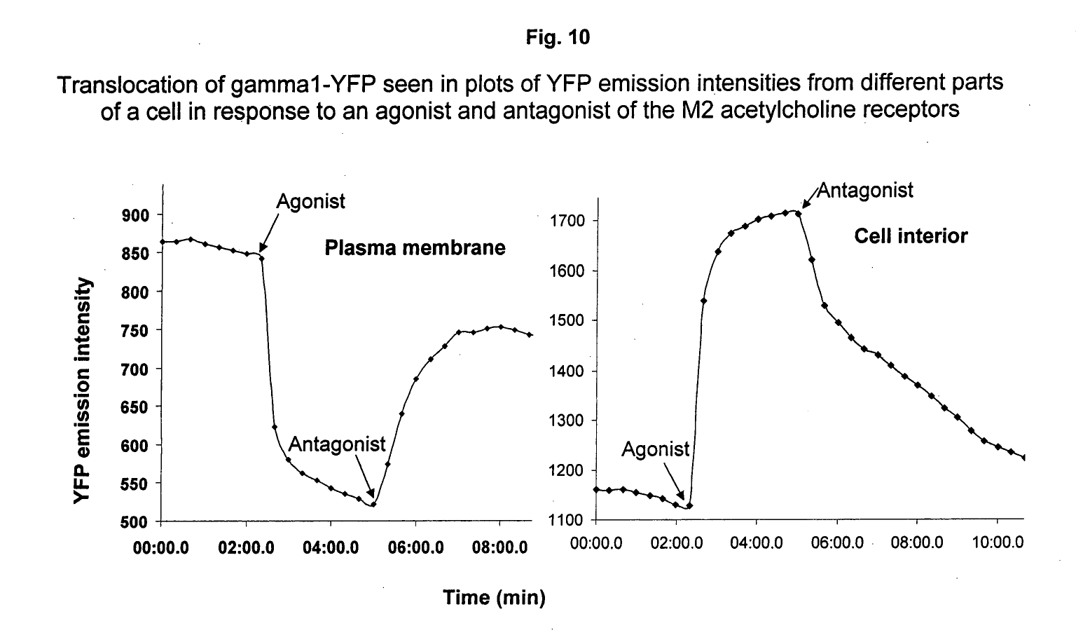

Plots of the fluorescence intensity from the YFP tagged gamma1 subunit in the same experiment show the translocation of the protein in response to the agonist and antagonist (FIG. 10).

### Example 8

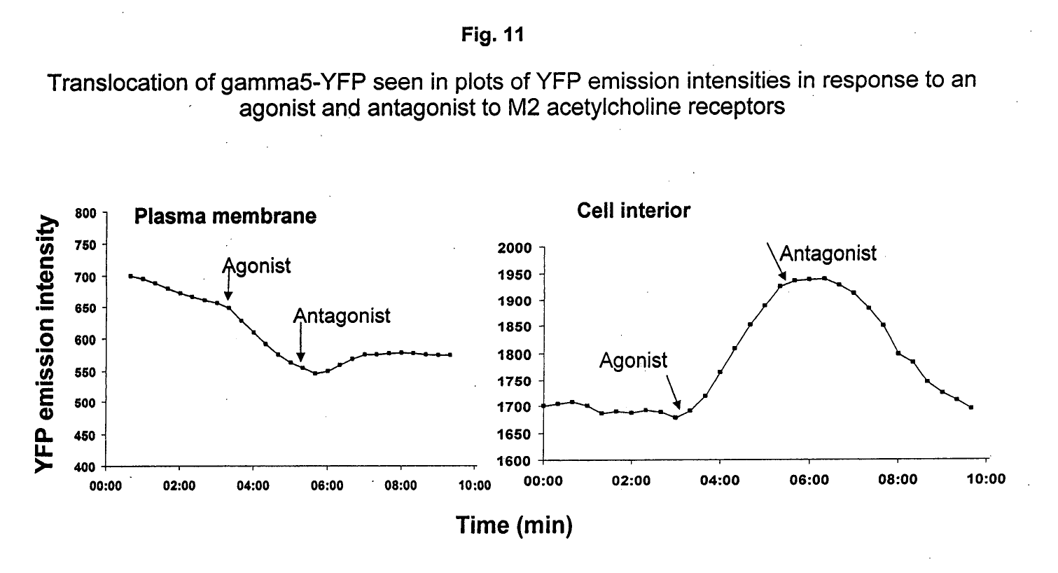

Biosensor cells coexpressing another gamma subunit type (gamma 5) tagged with YFP along with beta1 and alpha-o respond to the action of an agonist and an antagonist of the expressed M2 muscarinic receptors similar to biosensor cells expressing introduced gamma11 as previously established (FIG. 11).

### Example 9

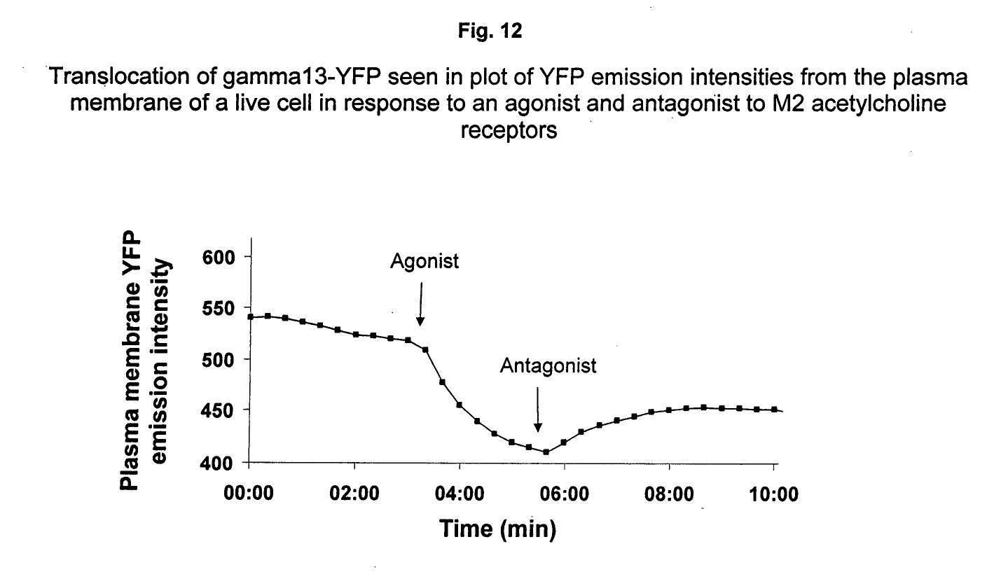

Biosensor cells coexpressing another gamma subunit type (gamma 13) tagged with YFP along with beta1 and alpha-o respond to the action of an agonist and an antagonist of the expressed M2 muscarinic receptors similar to biosensor cells expressing introduced gamma11 as previously established (FIG. 12).

The response of the biosensor cells to the action of an agonist and an antagonist on receptors in the cells expressing one of the introduced (transfected) gamma subunit types among the various gamma subunit types establishes the ability of various gamma subunit types belonging to the family of G protein gamma subunit types to translocates in the biosensor cell.

### Example 10

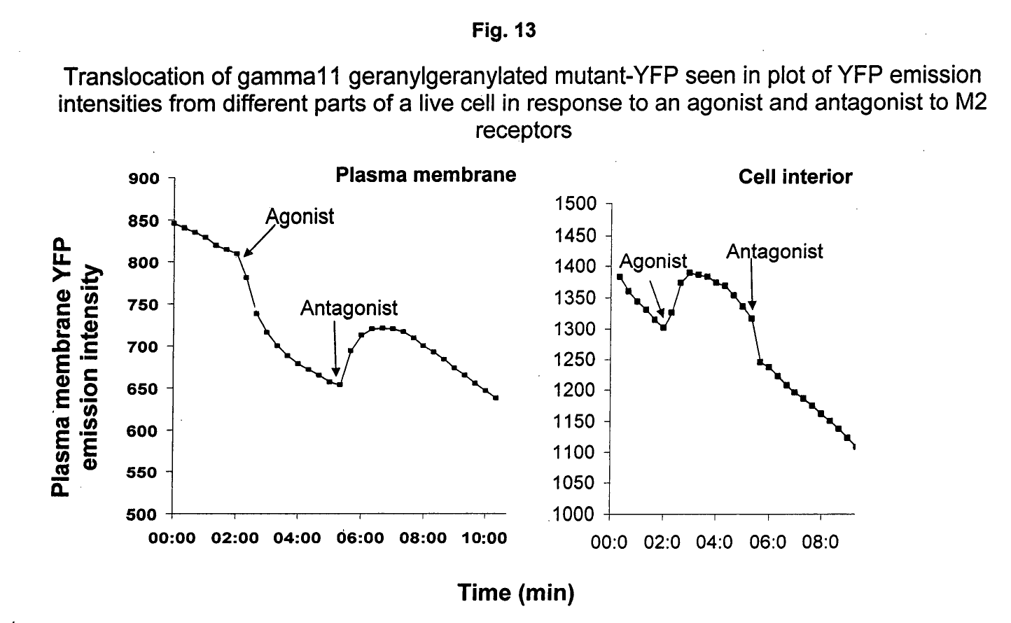

Biosensor cells coexpressing a mutant gamma11 subunit type tagged with YFP along with beta1 and alpha-o such that the mutant protein was geranylgeranylated instead of farnesylated respond to the action of an agonist and an antagonist of the expressed M2 muscarinic receptors similar to biosensor cells expressing introduced gamma11 as previously established (FIG. 13).

### Example 11

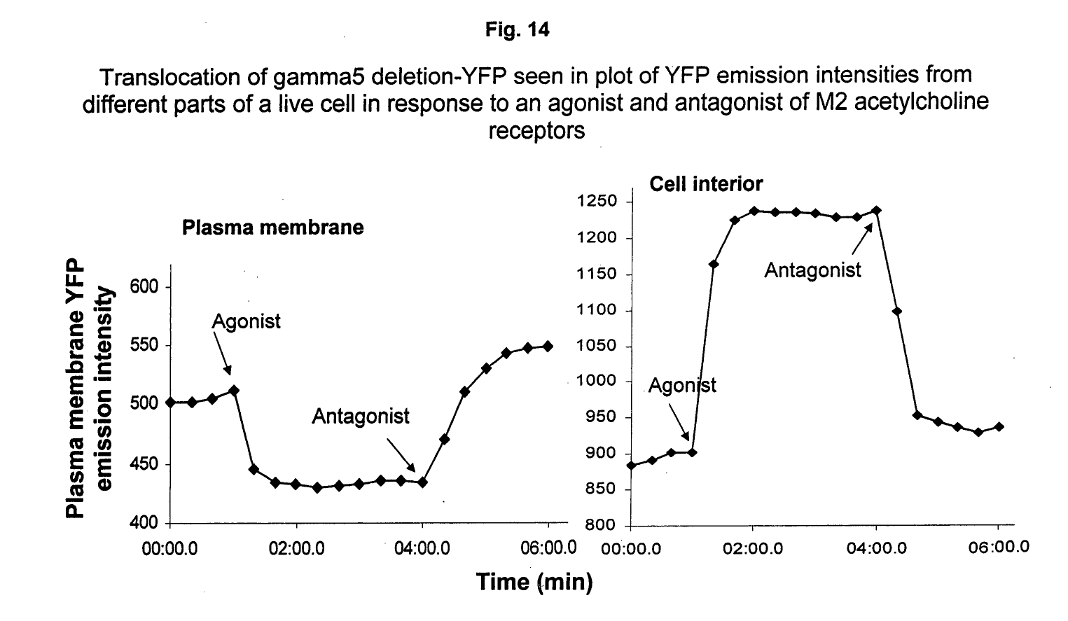

Biosensor cells coexpressing a mutant gamma5 subunit type tagged with YFP along with beta1 and alpha-o such that in the mutant protein ten residues upstream of the C terminal Cys residue were deleted respond to the action of an agonist and an antagonist of the expressed M2 muscarinic receptors similar to biosensor cells expressing introduced gamma11 as previously established (FIG. 14).

### Example 12

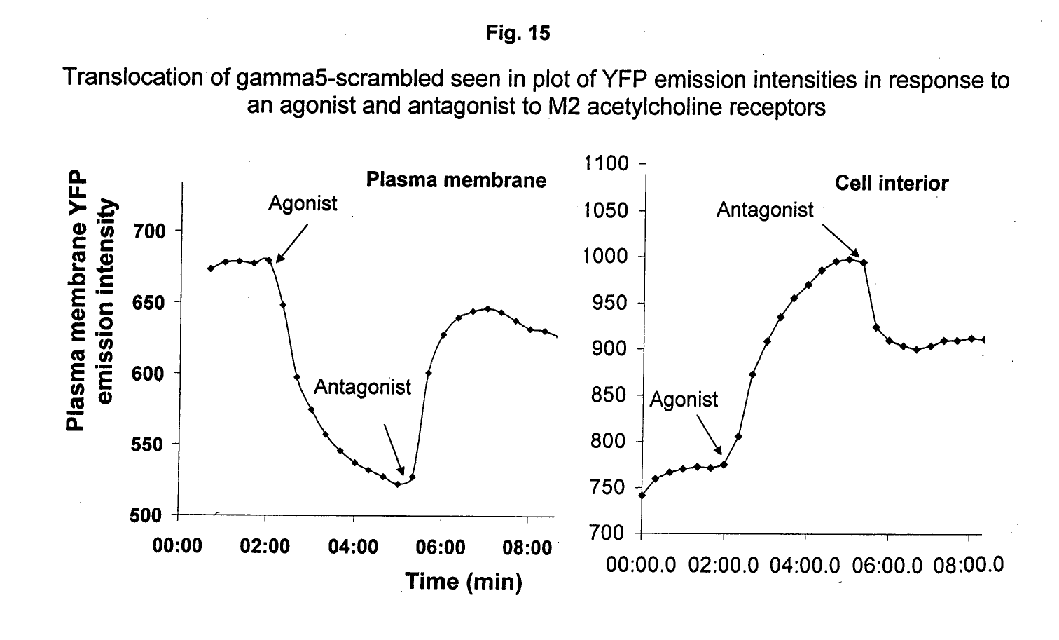

Biosensor cells coexpressing a mutant gamma5 subunit type tagged with YFP along with beta1 and alpha-o such that in the mutant protein ten residues upstream of the C terminal Cys residue were scrambled respond to the action of an agonist and an antagonist of the expressed M2 muscarinic receptors similar to biosensor cells expressing introduced gamma11 as previously established (FIG. 15).

### Example 13

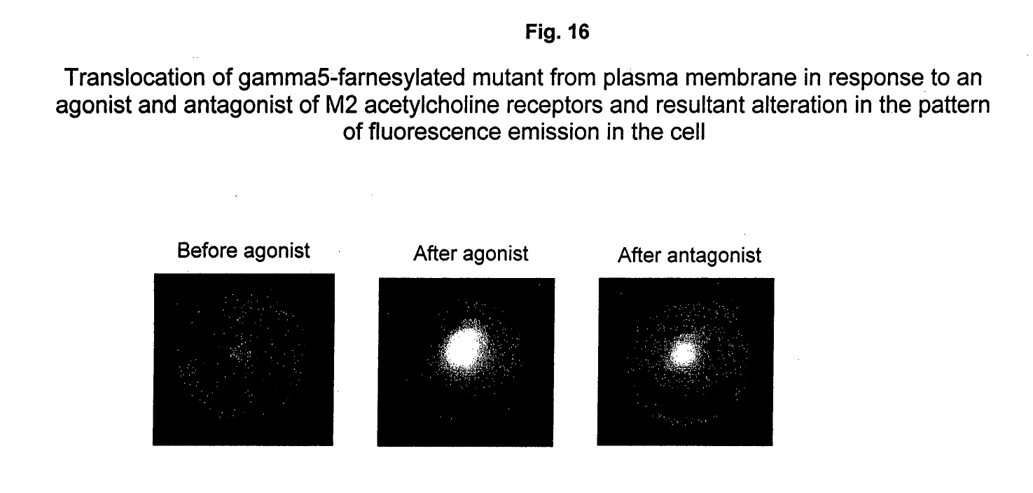

Single cell images of biosensor cells coexpressing the YFP tagged gamma5 subunit mutant which is famesylated with beta1 and alpha-o showing the translocation of the mutant farnesylated gamma5 subunit in response to the action of an agonist and an antagonist of the expressed M2 muscarinic receptors similar to biosensor cells expressing the gamma11 subunit (FIG. 16).

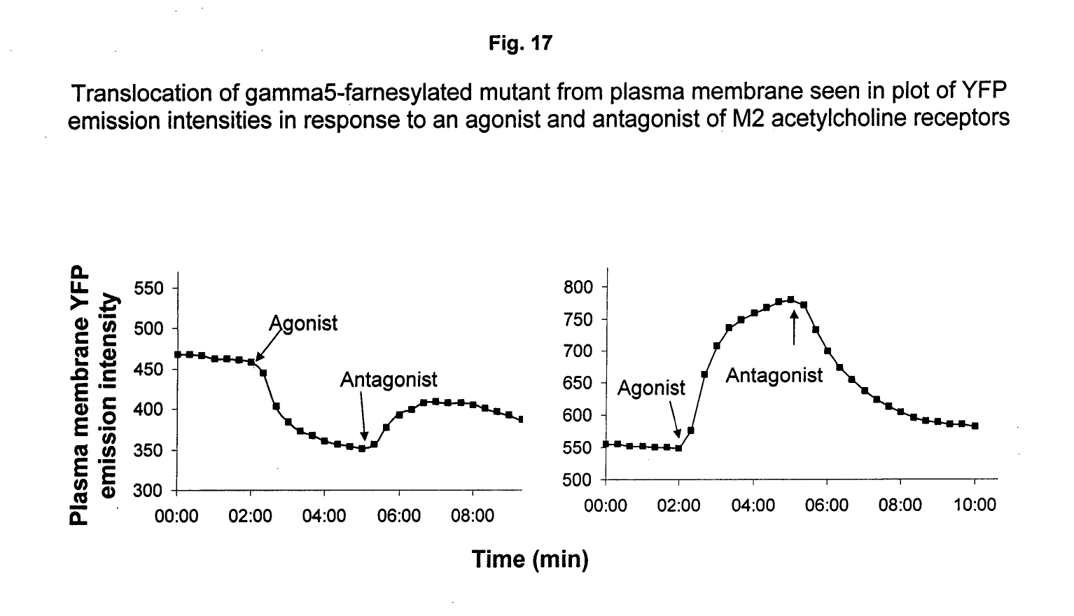

A plot of the fluorescence intensity from biosensor cells coexpressing the YFP tagged gamma5 subunit mutant which is farnesylated along with beta1 and alpha-o showing the translocation of the mutant farnesylated gamma5 subunit in response to the action of an agonist and an antagonist of the expressed M2 muscarinic receptors similar to biosensor cells expressing the gamma11 subunit (FIG. 17).

Thus the translocation process can be influenced by both the C terminal amino acid sequence of the gamma subunit types and the type of prenyl moiety attached to the C terminal tail of gamma subunits.

Gamma subunits mutants with alteration sat the C terminus can therefore be used to increase or decrease the extent of translocation in response to receptor activity.

### Example 14

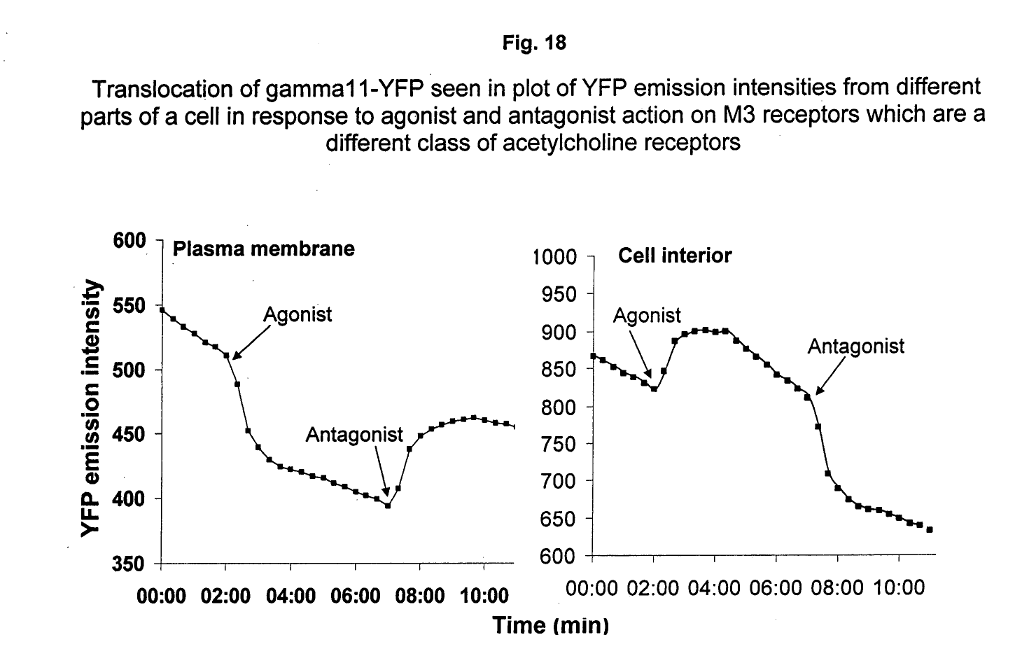

Biosensor cells coexpressing the gamma11 subunit type tagged with YFP along with beta1 and an alpha-o alpha-q chimera that contained the C terminal eleven residues of alpha-q replacing the corresponding sequence of alpha-o respond to the action of an agonist and an antagonist of the expressed M3 muscarinic receptors similar to biosensor cells expressing the related but distinct receptor type M2 receptors as previously established (FIG. 18).

The Go biosensor properties can thus be altered dramatically by substituting the C terminal domain of alpha-o-CFP in the biosensor with the C terminal domain of alpha-q. The resultant Go-q sensor is not activated by the M2 muscarinic receptor unlike the Go biosensor.

The Go-q biosensor was activated in an enhanced fashion compared to the Go biosensor by the M3 muscarinic receptor, a receptor type that normally couples to Gq type G proteins. The Go-q biosensor contains alpha-o-q-CFP that is an altered form of alpaha-o-CFP in which the C terminal domain of alpha-o was substituted with the C terminal domain of alpha-q.

Mutant G protein sensors with different C terminal domains can thus be used to specify coupling to different receptor types and can be used to both identify as well as classify candidate therapeutic molecules that bind to these different types of receptors.

### Example 15

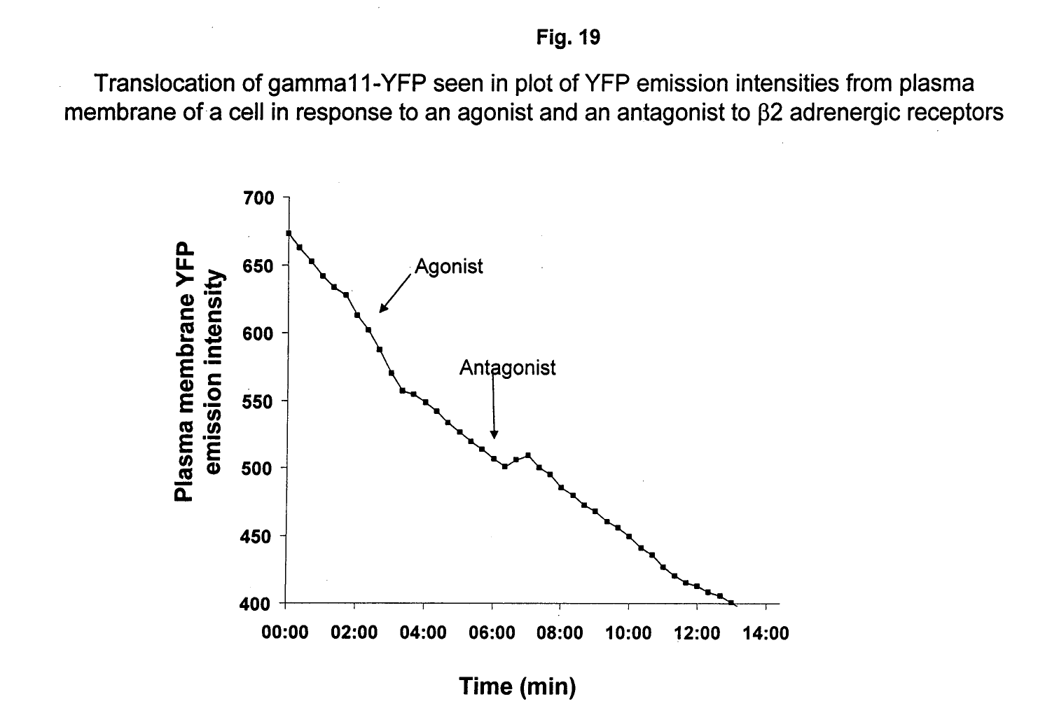

Biosensor cells coexpressing the gamma11 subunit type tagged with YFP along with alpha-o-CFP and beta1 respond to the action of an agonist and an antagonist of stably expressed beta2 adrenergic receptors in CHO cells (FIG. 19) similar to biosensor cells expressing the unrelated and distinct receptor types, M2, M3 and 5HT receptors as previously established.

The sensor thus responds in terms of translocation with all three G protein coupled receptor classes, Gi/o, Gq and Gs.

### Example 16

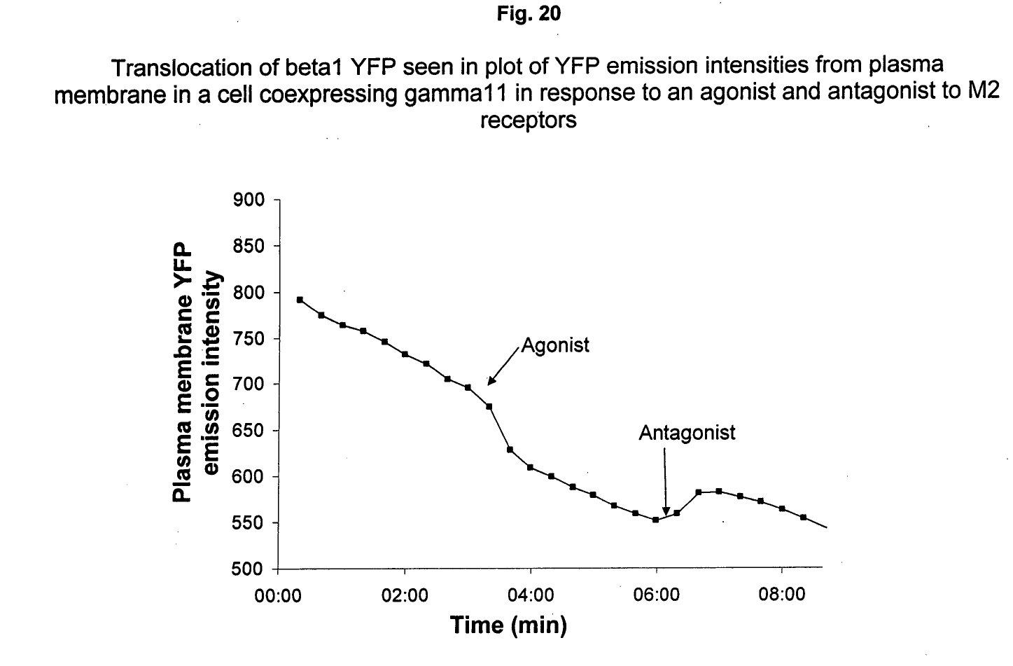

Biosensor cells coexpressing a beta1 tagged with YFP along with gamma11 and alpha-o respond to the action of an agonist and an antagonist of the expressed M2 muscarinic receptors similar to biosensor cells expressing introduced YFP tagged gamma11 as previously established (FIG. 20).

The response of the beta1 subunit indicates that it is translocatable in response to agonist and antagonist action on the biosensor cells.

The response of beta1 indicates that the translocation of the beta subunit can also be used to measure the action of agonist, antagonist, inverse agonist or allosteric regulator of the receptors on biosensor cells.

### Example 17

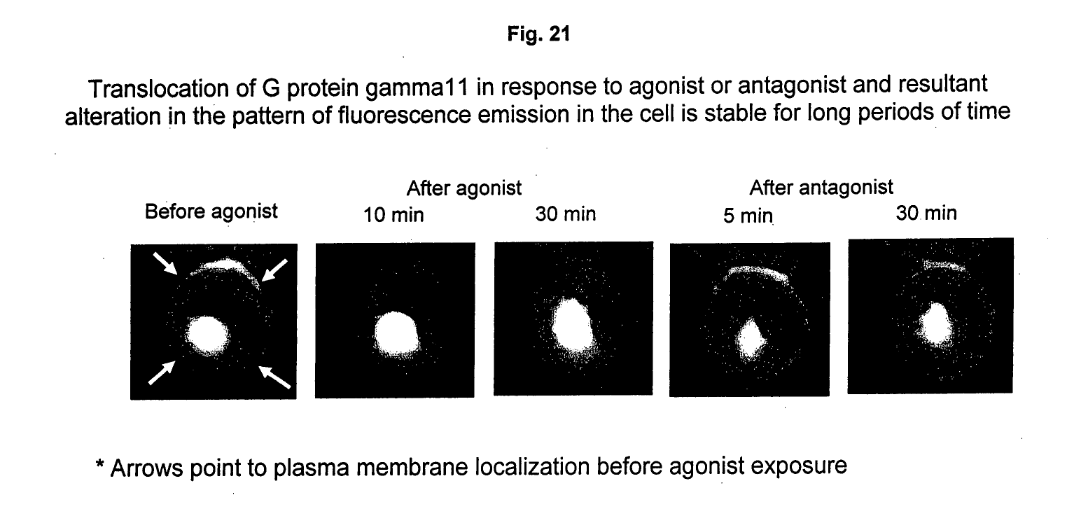

Single cell images of the responses of the biosensor cell to agonist and antagonist are shown to be stable over relatively long periods of time since the translocation of the YFP tagged gamma11 from plasma membrane to cell interior is retained over 30 min and the translocation of the YFP tagged gamma11 back to the plasma membrane from the cell interior is retained over 30 min also (FIG. 21).

The ability to retain the altered distribution of the fluorescent bisosensor for these long periods of time establishes the validity of using the methods described in FIG. 1 and FIG. 2 to perform high throughput screening of candidate therapeutic molecules because these methods will require relatively short periods of time well within the time frame of image pattern stability to acquire the images necessary for processing.

### Example 18

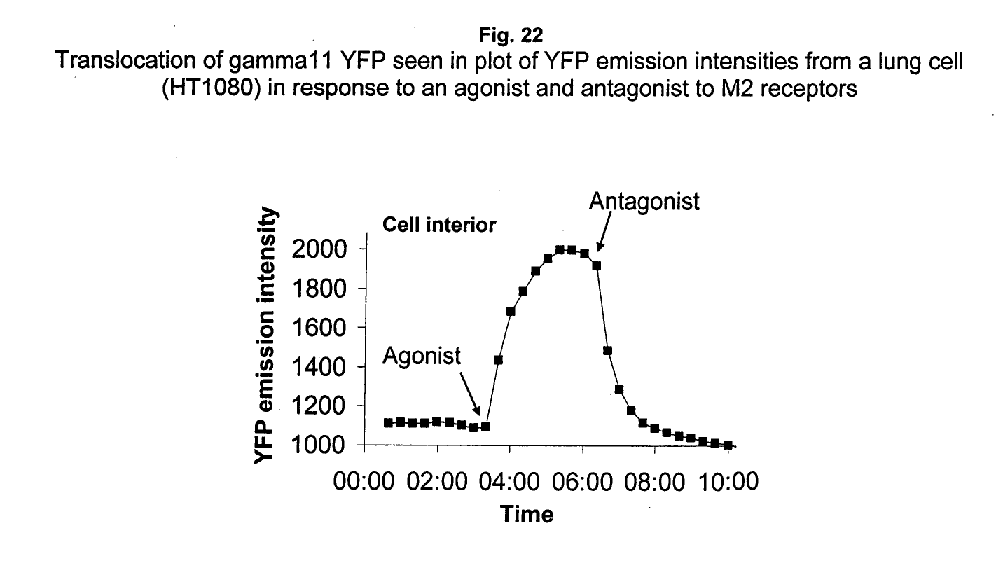

Biosensor cells comprising a distinctly different cell line from human lungs, HT1080, coexpressing a gamma11 tagged with YFP along with beta1 and alpha-o respond to the action of an agonist and an antagonist of the expressed M2 muscarinic receptors (FIG. 22) similar to biosensor cells comprising M2-CHO cells expressing introduced YFP tagged gamma11 as previously established (FIG. 4).

The response of the gamma11 subunit in a distinctly different cell line from a different mammalian species indicates that it is translocatable in response to agonist and antagonist action in different kinds of mammalian cell types.

### Example 19

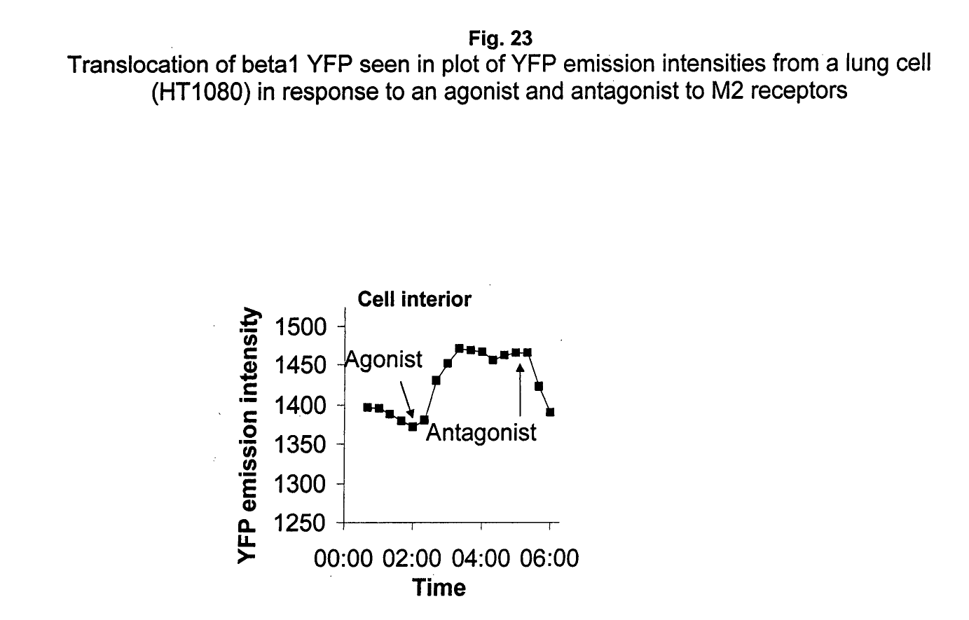

Biosensor cells comprising a distinctly different cell line from human lungs, HT 1080, coexpressing a beta1 tagged with YFP along with alpha-o respond to the action of an agonist and an antagonist of the expressed M2 muscarinic receptors (FIG. 23) similar to biosensor cells comprising M2-CHO cells expressing introduced YFP tagged beta1 with gamma11 as previously established (FIG. 20).

The response of beta1 in the absence of introduced gamma subunit indicates that the translocation of the beta subunit can also be used to detect the action of agonist, antagonist, inverse agonist or allosteric regulator of receptors on biosensor cells.

Examples (1-19) demonstrate that the expressed G protein biosensor containing various gamma subunit types and mutants that modified the gamma subunit amino acid sequence and/or the post translational modification were effectively operated with different receptor types that were both endogenous and introduced.

Examples (1-19) demonstrate that the G protein biosensor identified specific candidate molecules acting on particular receptors thus establishing a linkage between candidate molecules and associated receptors. This shows that the biosensor cell provides the capability to de-orphan receptors.

Advantageously, the functional cell based high throughput assay satisfies the ever growing demand for a biosensor that identifies and categorizes candidate therapeutic drugs from among candidate drugs collections/libraries in a a very rapid, highly sensitive, non-invasive assay. Candidate drugs refers to these drugs/molecules for which an identification and classification or re-classification is desired.

The assay is highly sensitive and will measure relatively low concentrations of candidate molecules conserving expensive compounds. FIG. 3 shows the biosensor cell responding to 10 nM agonist.

The assay is very rapid. FIG. 4 shows the biosensor cell responding with translocation to both agonist and antagonist within 20 sec.

Advantageously the biosensor cell is useful to provide a screening method for determining therapeutic candidate drugs from among candidate drugs. As used herein the term “candidate therapeutic drug” refers to a drug which has shown activity in a G protein biosensor as an agonist, antagonist or inverse agonist. It is particularly desired to now have the classification system and method for such drugs provided in this invention, including the capability to decide whether to advance a drug to a second level in evaluation such as to advance a drug to secondary screening or advance a drug for testing presently in secondary screening to tertiary screening. The biosensor cell is particularly useful in the increasingly central technology in research and development of better medicines for mankind.

Advantageously use of the biosensor cell provides a non-invasive method which does not disrupt the cell for assaying receptor activity and considerably hastens the process of drug discovery by facilitating the rapid screening of a large library of candidate molecules with a large array of receptor types to classify those molecules which should be further tested or moved further along the research pipeline toward commercialization or in an aspect, those molecules on which further testing should be deferred.

This novel G protein based biosensor cell provides non-invasive rapid screening of candidate drug molecules targeted at G protein coupled receptors in a reproducible and unambiguous fashion. The biosensor cell allows the detection, observation and measurement of signaling properties and dynamics in an on line living intact cells utilizing proteins with none, substantially none or minimum disruption to native cellular signaling networks.

Additionally this invention provides receptor stimulated G proteins and a non-invasive non-destructive method (model) of screening candidate molecules using the same live cell biosensor cell to identify candidate therapeutic drug molecules from among candidate molecules.

In an aspect, this invention provides a method to identify those candidate molecules which are not therapeutic drug molecules, which in today's world is an ever increasing desired method. It is highly desired to identify the molecules for which research is to continue as well as those for which research is to stop. This invention permits the prioritization of drug candidates based on their performance/evaluation in a biosensor cell.

Also this invention provides receptor stimulated G proteins having subunits respectively fused with a fluorescent or luminescent protein useful in live extraordinarily complex mammalian cells in a biological system having large number of signaling pathways to screen for and to identify therapeutic candidates.

This invention is useful as a tool to identify and/or classify molecules as agonist, antagonist, inverse agonist or innocuous candidate drug molecules of therapeutic value for use in research, industrial and commercial environments and to identify and classify molecules that bind to uncharacterized mammalian orphan G protein coupled receptors.

This invention is also useful as a tool to obtain information about both the temporal and spatial changes in biosensor activity in an intact living cell elicited by candidate therapeutic molecules directed at specific receptors.

This invention is also useful as a tool to identify and/or classify candidate molecules of therapeutic value as agonist, antagonist or inverse agonists of receptors using high content screening.

In an aspect, therapeutic molecules include small molecules that are pharmaceutical drugs, vaccines, medicines and antibiotics which generally provide a beneficial value to a patient (human or other primate) taking one or more and in need of treatment for a particular medical affliction.

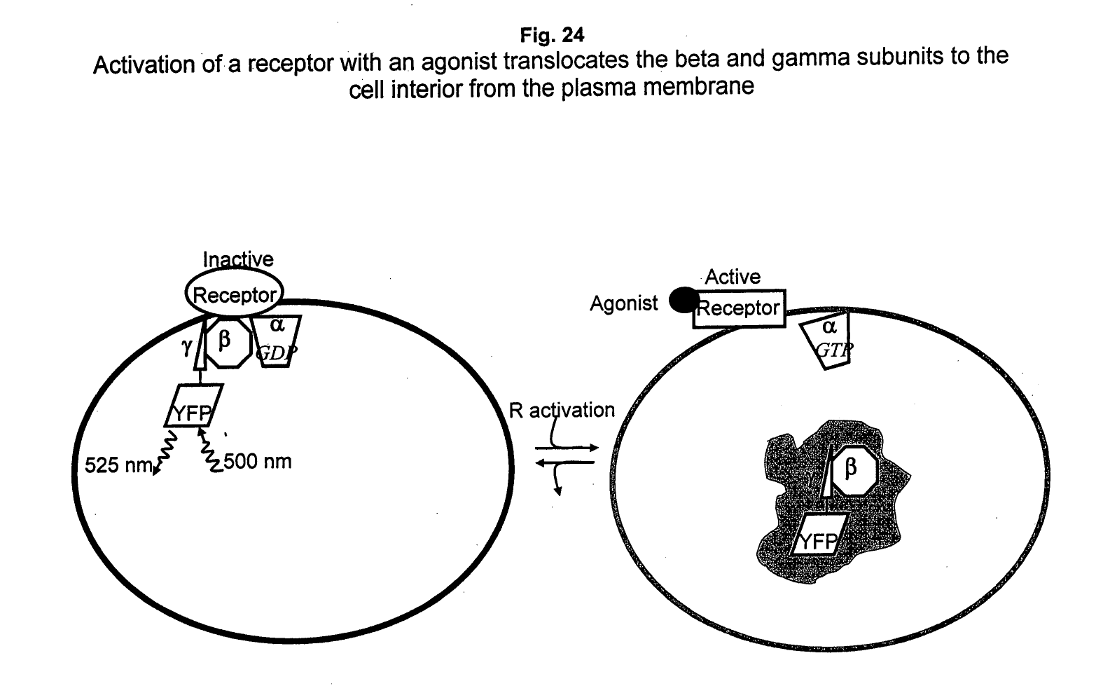

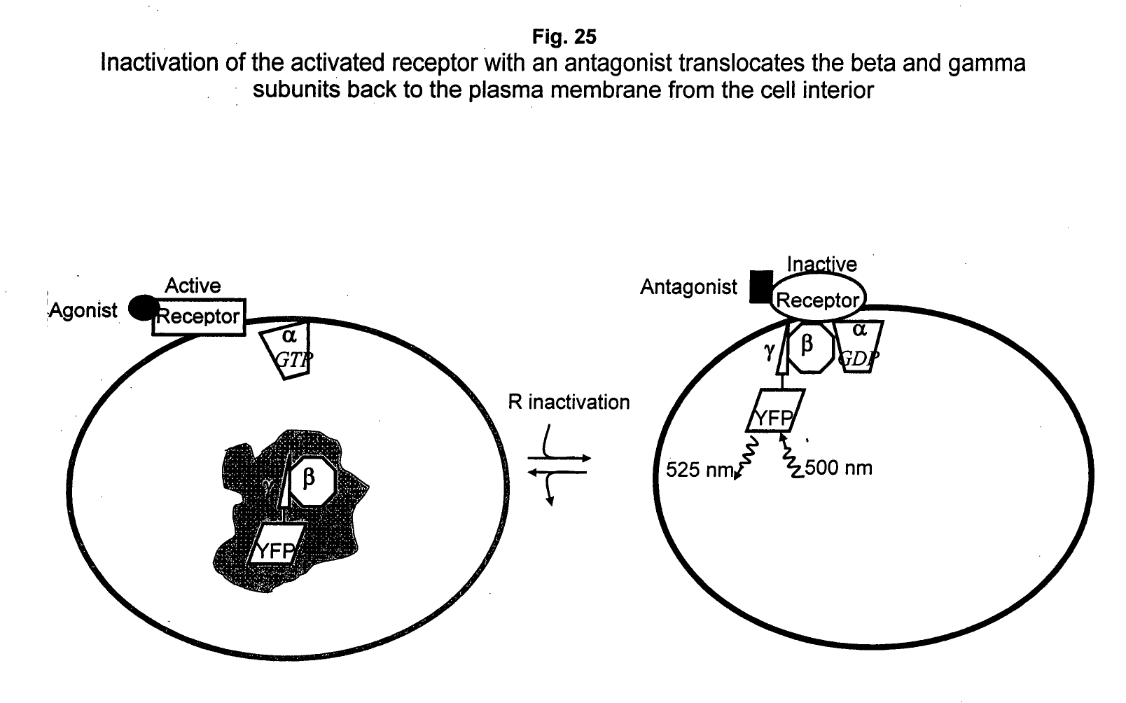

FIG. 24 and FIG. 25 are diagrammatic representations of a G protein biosensor comprising alpha, beta and gamma subunits wherein in this aspect presented the gamma subunit is tagged with a fluorescent protein, YFP. The process of receptor activation and inactivation of this sensor with the resultant translocation of the sensor from one part of the cell to the other are shown.

While the invention has been described in terms of various specific embodiments, those skilled in the art will recognize that the invention can be practiced with modification.

1. A functional biosensor comprising heterotrimeric G protein alpha, translocatable beta or translocatable gamma or translocatable beta and gamma subunits wherein at least the beta, gamma, or both beta and gamma subunits are tagged with a fluorescent protein or a luminescent protein.

2. A biosensor wherein said subunits comprise heterotrimeric G protein subunits capable of being activated by G protein coupled receptors.

3. A biosensor wherein either the beta subunit or the gamma subunit or both subunits are tagged with a fluorescent protein and the translocation of the fluorescent signal emission is observed.

4. A live functional G protein biosensor cell expressing endogenous G protein coupled receptors comprising a G protein alpha subunit that is endogenous or introduced into the cell, a beta subunit that is endogenous or introduced into the cell and an introduced gamma subunit tagged with a fluorescent protein.

5. A live functional G protein biosensor cell expressing endogenous G protein coupled receptors comprising a G protein alpha subunit that is endogenous or introduced into the cell, a gamma subunit that is endogenous or introduced into the cell and an introduced beta subunit tagged with a fluorescent protein.

6. A live functional G protein biosensor cell expressing introduced G protein coupled receptors comprising a G protein alpha subunit that is endogenous or introduced into the cell, a beta subunit that is endogenous or introduced into the cell and an introduced gamma subunit tagged with a fluorescent protein.

7. A live functional G protein biosensor cell expressing introduced G protein coupled receptors comprising a G protein alpha subunit that is endogenous or introduced into the cell, a gamma subunit that is endogenous or introduced into the cell and an introduced beta subunit tagged with a fluorescent protein.

8. A method for screening natural or chemically synthesized candidate agonists, antagonists, inverse agonists, allosteric regulators and other molecules that bind to previously characterized, uncharacterized or “orphan” G protein coupled receptors, by operating an intact living cell containing said receptors and G protein alpha, beta and gamma subunits wherein beta, gamma or both subunits are tagged with a fluorescent protein by exposing to the said candidate agonists to elicit translocation of the fluorescent signal from plasma membrane to the interior of the cell and subsequently exposing to a candidate antagonist or inverse agonist to elicit translocation of the fluorescent signal from the cell interior to the plasma membrane thereby identifying candidate agonist(s), antagonist(s) and inverse agonist(s) for said characterized, uncharacterized or orphan receptor.

9. A method for screening natural or chemically synthesized candidate inverse agonists, allosteric regulators and other molecules that bind to previously characterized, uncharacterized or “orphan” G protein coupled receptors, by operating the aforementioned biosensor cells to an agonist to elicit translocation of the fluorescent signal from plasma membrane to the interior of the cell and subsequently exposing to an antagonist to elicit translocation of the fluorescent signal from the cell interior to the plasma membrane and comparing these images with images of another such biosensor cell exposed to an agonist in the presence of a candidate allosteric regulator and antagonist in the presence of a candidate allosteric regulator to identify whether the candidate allosteric regulator has an effect on the agonist, antagonist or inverse agonist activity thereby classifying it as an allosteric regulator.

10. A biosensor cell wherein said living cell comprises receptors and G protein biosensor.

11. A method for determining G protein coupled receptor regulated signal transduction activity in an intact living cell using the biosensor cell to quantifiably measure G protein receptor signaling activity non-invasively.

12. A biosensor cell wherein said living cell comprises receptors and G protein biosensor.

13. A non-invasive method for identifying a candidate therapeutic drug molecule, which comprises obtaining images of a biosensor cell over a time period from a live biosensor cell expressing a characterized receptor with a known ligand or an orphan receptor with unknown ligand (a) in the absence of an added candidate molecule, (b) in the presence of an added molecule and then comparing said images (b) with said images (a) to obtain a comparison of the images of (b) with the images of (a).

14. A biosensor cell wherein said living cell comprises receptors and G protein biosensor.

15. A method wherein if said comparison shows emitted fluorescence signal intensity on the plasma membrane after the addition of a candidate molecule (b) is less than the fluorescence signal intensity on the plasma membrane before the addition of the candidate (a) and emitted fluorescence signal intensity in the cell interior after the addition of a candidate molecule (b) is more than the fluorescence signal intensity in the cell interior before the addition of the candidate (a) indicating translocation of the fluorescent signal, then one classifies the molecule as an agonist candidate therapeutic drug molecule. If the comparison shows that said images (b) is similar to said images (a), then one classifies the molecule as a molecule likely not having agonistic therapeutic value.

16. A method wherein a number of different molecules are added to said biosensor cells, singly or as a pool of various candidate molecules and images of these candidate molecules are obtained to classify candidate therapeutic molecules.

17. A non-invasive screening method for identifying agonist candidate therapeutic drug molecules using an intact live biosensor cell system containing a receptor and a G protein biosensor, which when exposed to a candidate molecule results in translocation of the said fluorescence signal from the plasma membrane to the cell interior indicating that said candidate is an agonist therapeutic drug molecule.

18. A biosensor cell wherein said living cell comprises receptors and G protein biosensor.

19. A non-invasive screening method for identifying antagonistic activity of a candidate therapeutic drug molecule using an intact live biosensor cell, wherein the cell is first exposed to a known agonist and subsequently to a candidate therapeutic drug molecule, said agonist being capable of translocating the fluorescent signal from the plasma membrane to the cell interior on binding the receptor, and candidate antagonist being capable of inducing the translocation of the fluorescent signal back to the plasma membrane from the cell interior indicating that said candidate is a therapeutic antagonist molecule.

20. A method wherein a known agonist is applied to the biosensor cells to obtain images (c) and subsequently adding to biosensor cells a candidate therapeutic antagonist molecule which provides images (d) and comparing the images (d) with the images (c).

21. A biosensor cell wherein the living cell comprises receptors and G protein biosensor.

22. A method wherein if the fluorescence signal in images (d) after the addition of a candidate antagonist molecule shows the translocation of the fluorescence signal from cell interior to the plasma membrane compared to the images (c) after the addition of the known agonist, then one classifies the molecule added second as an antagonist candidate therapeutic drug molecule.

23. A method wherein if the fluorescence signal in images (d) after the addition of a candidate antagonist molecule does not show any changes in comparison to the images (c) after the addition of the known agonist, then one classifies the molecule added second as innocuous in terms of antagonist activity.

24. A biosensor cell wherein said live cell comprises receptors and G protein biosensor.

25. A non-invasive screening method for identifying natural or chemically synthesized candidate agonists and antagonists that bind to uncharacterized or “orphan” mammalian receptors thus de-orphaning orphan receptors, said method comprising exposure of the biosensor cell to candidate agonist and antagonist molecules and identifying agonists first and antagonists subsequently based on the ability of the candidate molecules to induce translocation of the fluorescent signal on binding to the receptor.

26. A method wherein a number of different molecules are added to the biosensor containing cells, singly or as a pool of various candidate molecules and images of the cells exposed to these candidate molecules are obtained to classify candidate therapeutic molecules.

27. A method for identifying a candidate therapeutic molecule as an inverse agonist by obtaining a images of biosensor cells containing overexpressed or mutant receptors of defined or orphan status possessing constitutive activity such that the images of cells (e) after exposure to the candidate inverse agonist molecule when compared to the images of cells without any exposure to any molecule that binds the receptor (a) indicate translocation of the fluorescent signal from cell interior to the plasma membrane allowing for the classification of the molecule as an inverse agonist.

28. A method wherein if addition of the candidate does not alter the images (e), then the added molecule is classified as innocuous in terms of inverse agonist activity.

29. A method wherein a number of different molecules are added to the biosensor containing cells, singly or as a pool of various candidate molecules and images of the cells exposed to these candidate molecules are obtained to classify candidate therapeutic molecules.

30. A live functional biosensor cell comprising a G protein alpha subunit in which its carboxyl-terminal domain has been substituted with the corresponding domain of another alpha subunit with a distinctly different receptor specificity such that the biosensor cell can be used for screening for therapeutic molecules that are agonists, antagonists, inverse agonists or allosteric regulators of different receptor types.

31. A live functional biosensor cell containing mutant forms of the G protein sensor that alter the receptor coupling capability of the G protein such that it can be used for identifying and classifying therapeutic molecules which are agonists, antagonists, inverse agonists or allosteric regulators of various receptor types.

32. A method for increasing the number of receptor types that will couple to the biosensor by mutationally altering the C terminal tail of the alpha subunit constituent of the biosensor.

33. A method for altering the intensity of the translocation response from biosensor cells by mutationally altering the alpha subunit.

34. A method for altering the intensity of the translocation response from biosensor cells by using particular alpha subunit types.

35. A method for altering the intensity of the translocation response from biosensor cells by mutationally altering the gamma subunit.

36. A method for altering the intensity of the translocation response from biosensor cells by using particular gamma subunit types.

37. A method for altering the intensity of the translocation response from biosensor cells by mutationally altering the beta subunit.

38. A method for altering the intensity of the translocation response from biosensor cells by using particular beta subunit types.

39. A live functional G protein biosensor cell expressing introduced G protein alpha subunit fused to a G protein coupled receptor and a beta or gamma or both beta and gamma subunits, wherein the beta or gamma or both beta and gamma subunits are tagged to a protein that is fluorescence or luminescence capable and the addition of an agonist for the tethered receptor induces translocation of the beta, gamma or both subunits to the cell interior from the plasma membrane and the addition of an antagonist induced the translocation of the beta or gamma or beta and gamma subunits back to the plasma membrane.

40. A method for identifying and classifying multiple candidate therapeutic molecules using the same G protein biosensor cell by repetitive treatment with candidate agonist, antagonist, inverse agonist and allosteric regulator molecules.

41. A method for identifying and classifying a single candidate therapeutic molecule using the same G protein biosensor cell by repetitive treatment with candidate therapeutic molecules of agonist, antagonist, inverse agonist and allosteric regulator or properties.

42. A method for identifying and classifying candidate therapeutic molecules which are agonists, antagonists, inverse agonists or allosteric regulators of various receptor types by performing high content screening of biosensor cells wherein “high content” is defined as information about biosensor activity in terms of both time dependence and spatial location in an intact cell maintaining structural and functional integrity.

43. A method for identifying and classifying candidate therapeutic molecules which are agonists, antagonists, inverse agonists or allosteric regulators of various receptor types that have specific effects on cellular components including plasma membrane, intracellular organelles and cytosol using the intact, functional G protein biosensor cell.

44. A method of classifying candidate therapeutic molecules as agonists, antagonists, inverse agonists or allosteric regulators using biosensor cells and screening for predicted changes in the images from these cells in response to the addition of the candidate molecules.


SEQUENCE LISTING (8 sequences)

SEQ ID NO: 1 - protein, 5 aa, Artificial Sequence
  Description of Artificial Sequence Synthetic peptide
TGVSS

SEQ ID NO: 2 - protein, 4 aa, Unknown Organism
  Description of Unknown Organism Amino acid segment from gamma5
CSFL

SEQ ID NO: 3 - protein, 16 aa, Unknown Organism
  Description of Unknown Organism Amino acid segment from gamma5
TGVSSSTNPFRPQKVC

SEQ ID NO: 4 - protein, 14 aa, Artificial Sequence
  Description of Artificial Sequence Synthetic peptide
TPVNFSQVSKCSFL

SEQ ID NO: 5 - protein, 14 aa, Unknown Organism
  Description of Unknown Organism Amino acid segment from gamma5
STNPFRPQKVCSFL

SEQ ID NO: 6 - protein, 11 aa, Artificial Sequence
  Description of Artificial Sequence Synthetic peptide
LQLNLKEYNLV

SEQ ID NO: 7 - protein, 11 aa, Unknown Organism
  Description of Unknown Organism Amino acid segment from alpha-o protein
IANNLRGCGLY

SEQ ID NO: 8 - protein, 4 aa, Artificial Sequence
  Description of Artificial Sequence Amino acid segment from gamma11
CVIS